SEQUENCE LISTING (6 sequences)

SEQ ID NO: 1 - protein, 386 aa, Human
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 2 - DNA, 3089 bp, Human
agcagttcctcctaactcctgccagaaacagctctcctcaacatgagagctgcacccctcctcctggccagggcagcaagccttagccttggcttcttgtttctgctttttttctggctagaccgaagtgtactagccaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaattccagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatggaattcacaagcagaaagagaaatctaggctccaagggggtgtcctggtcaatgaaatcctcaatcacatgaagagagcaactcagataccaagctacaaaaaacttatcatgtattctgcgcatgacactactgtgagtggcctacagatggcgctagatgtttacaacggactccttcctccctatgcttcttgccacttgacggaattgtactttgagaagggggagtactttgtggagatgtactaccggaatgagacgcagcacgagccgtatcccctcatgctacctggctgcagccccagctgtcctctggagaggtttgctgagctggttggccctgtgatccctcaagactggtccacggagtgtatgaccacaaacagccatcaaggtactgaggacagtacagattagtgtgcacagagatctctgtagaaagagtagctgccctttctcagggcagatgatgctttgagaacatactttggccattaccccccagctttgaggaaaatgggctttggatgattattttatgttttagggacccccaacctcaggcaattcctacctcttcacctgaccctgcccccacttgccataaaacttagctaagttttgttttgtttttcagcgttaatgtaaaggggcagcagtgccaaaatataatcagagataaagcttaggtcaaagttcatagagttcccatgaactatatgactggccacacaggatcttttgtatttaaggattctgagattttgcttgagcaggattagataagtctgttctttaaatttctgaaatggaacagatttcaaaaaaaattcccacaatctagggtgggaacaaggaaggaaagatgtgaataggctgatggggaaaaaaccaatttacccatcagttccagccttctctcaaggagaggcaaagaaaggagatacagtggagacatctggaaagttttctccactggaaaactgctactatctgtttttatatttctgttaaaatatatgaggctacagaactaaaaattaaaacctctttgtgtcccttggtcctggaacatttatgttccttttaaagaaacaaaaatcaaactttacagaaagatttgatgtatgtaatacatatagcagctcttgaagtatatatatcatagcaaataagtcatctgatgagaacaagctatttgggcacaacacatcaggaaagagagcaccacgtgatggagtttctccagaagctccagtgataagagatgttgactctaaagttgatttaaggccaggcatggtggtttacgcctataatcccagcattttgggactccgaggtgggcagatcacttgagctcaggagctcaagatcagcctgggcaacatggtgaaaccttgtctctacataaaatacaaaaacttagatgggcatggtgctgtgtgcctatagtccactacttgtggggctaaggcaggaggatcacttgagccccggaggtcgaggctacagtgacccaagagtgcactactgtactccagccagggcaagagagcgagaccctgtctcaataaataaataaataaataaataaataaataaataaaaacaaagttgattaagaaaggaagtataggccaggcacagtggctcacacctgtaatccttgcattttggaaggctgaggcaggaggatcactttaggcctggtgtgttcaagaccagcctggtcaacatagtgagacactgtctctaccaaaaaaaggaaggaagggacacatatcaaactgaaacaaaattagaaatgtaattatgttatgttctaagtgcctccaagttcaaaacttattggaatgttgagagtgtggttacgaaatacgttaggaggacaaaaggaatgtgtaagtctttaatgccgatatcttcagaaaacctaagcaaacttacaggtcctgctgaaactgcccactctgcaagaagaaatcatgatatagctttccatgtggcagatctacatgtctagagaacactgtgctctattaccattatggataaagatgagatggtttctagagatggtttctactggctgccagaatctagagcaaagccatcccccctcctggttggtcacagaatgactgacaaagacatcgattgatatgcttctttgtgttatttccctcccaagtaaatgtttgtccttgggtccattttctatgcttgtaactgtcttctagcagtgagccaaatgtaaaatagtgaataaagtcattattaggaagttcaaaaaaaaaaaa

SEQ ID NO: 3 - protein, 144 aa, Human
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 4 - DNA, 767 bp, Human
cggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatagccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctaaaaaaaaaaaaa

SEQ ID NO: 5 - protein, 144 aa, Artificial Sequence; identical to SEQ ID NO: 3
  Made in a lab from human amino acids

SEQ ID NO: 6 - DNA, 767 bp, Artificial Sequence; identical to SEQ ID NO: 4
  Made in a lab from human nucleic acids

(19) United States
(12) Patent Application Publication
(10) Pub. No.: US 2004/0161413 A1
(43) Pub. Date: Aug. 19, 2004

(54) Immunotherapeutic compositions and methods for the treatment of moderately to well-differentiated cancers

(75) Inventors: Reiner Laus, Bellevue, WA (US); Mitchell H. Gold, Seattle, WA (US); Madhusudan Peshwa, Issaquah, WA (US); Grant Pickering, Seattle, WA (US); Jelle Kylstra, Issaquah, WA (US)

Correspondence Address: SPECKMAN LAW GROUP PLLC, 1501 WESTERN AVE, SEATTLE, WA, 98101, US

(73) Assignee: DENDREON CORPORATION, Seattle, WA (US)

(21) Appl. No.: 10/666,122

(22) Filed: Sep. 19, 2003

Related U.S. Application Data
(60) Provisional application No. 60/412,271, filed on Sep. 20, 2002.
(60) Provisional application No. 60/475,335, filed on Jun. 2, 2003.

(51) Int. Cl. A61K 39/00

(52) U.S. Cl. 424/93.7; 424/277.1

(57) ABSTRACT

Provided are immunotherapeutic compositions and methods for the treatment of cancers characterized by the presence of moderately to well-differentiated cancer cells. Exemplary immunotherapeutic compositions employ antigen presenting cells, including dendritic cells, activated by a protein conjugate such as fusion proteins including a prostatic acid phosphatase/granulocyte-macrophage colony stimulation factor fusion protein. Also disclosed are methods for assessing the susceptibility of cancer cells to treatment regimens employing one or more immunotherapeutic composition.

### RELATED APPLICATIONS

This application claims priority of U.S. Provisional Patent Application Serial No. 60/412,271, filed Sep. 20, 2002, and 60/475,355 filed Jun. 2, 2003.

### BACKGROUND OF THE INVENTION

1. Technical Field of the Invention

The present invention relates to compositions and methods for the treatment of cancer. More specifically, this invention provides immunotherapeutic compositions and methods for inhibiting the growth of cancer cells in a patient having a moderately to well-differentiated grade of cancer. In addition, the present invention further provides methods for assessing in a cancer patient the susceptibility of cancer cells to treatment regimens employing immunotherapeutic compositions.

2. Description of the Related Art

Cancer is a group of diseases characterized by uncontrolled growth and spread of abnormal cells and is the second leading cause of death in the United States, exceeded only by heart disease. About 1,268,000 new cancer cases were expected to be diagnosed and approximately 553,400 deaths were predicted to occur in 2001. In the United States, men have about a 1 in 2 lifetime risk of developing cancer, and for women, the risk is about 1 in 3.

Cancer of the prostate is among the most commonly diagnosed neoplasms in men in the United States. In 2001, an estimated 198,100 new cases of prostate cancer were diagnosed which represented ˜29% of all new cancer diagnoses in men. Approximately 31,500 deaths in 2001 were attributed to prostate cancer. Between 1988 and 1992, prostate cancer incidence rates increased dramatically, due to earlier diagnosis in men without any symptoms, through increased use of the prostate-specific antigen (PSA) blood test. Prostate cancer incidence rates subsequently declined and have leveled off. One in six men will develop prostate cancer at some point in his life.

Determining the appropriate therapy for any cancer requires an assessment of various factors that, in total, seek to predict the therapeutic efficacy of a given treatment modality. Conventional cancer treatment regimens depend upon the type of cancer, but often include surgical procedures, radiation therapy, chemotherapy, hormone therapy or a combination thereof.

In the specific case of prostate cancer, and in consideration of the patient's age, stage of cancer, and other medical conditions, surgery and/or radiation therapy are common approaches for treatment. Hormonal therapy and chemotherapy are frequently employed for metastatic disease. Hormone treatment may control prostate cancer for long periods by shrinking the size of the tumor thus relieving pain and other symptoms. In some instances, careful observation without immediate active treatment (“watchful waiting”) may be appropriate, particularly for older individuals with low-grade and/or early stage tumors.

More recently, advances in cancer research have yielded a variety of immunotherapeutic compositions for the treatment of cancers. Immunotherapeutic approaches to cancer treatment are based on the recognition that cancer cells can often evade the body's defenses against aberrant or foreign cells and molecules, and that these defenses might be therapeutically stimulated to regain lost ground. See, e.g., Klein, “Immunology,” pgs. 623-648 (Wiley-Interscience, New York, 1982).

Recent observations that various immune effectors can directly or indirectly inhibit growth of tumors have led to renewed interest in this approach to cancer therapy. Jager et al., Oncology 60 (1):1-7 (2001) and Renner et al., Ann Hematol 79(12):651-9 (2000). Modern immunotherapeutics include, for example, antibody-based therapeutics, polypeptide vaccines, and a number of cellular immunotherapeutics. Each of these immunotherapeutic treatment modalities has in common the enhancement of those components of the patient's immune system that are responsible for the surveillance and eradication of the cancer cells.

Regardless of the specific type of cancer, clinical outcome is frequently prognosticated by determining the differentiation state of the cancer cells. The relation between the extent of differentiation of a tumor and its biologic behavior has been known for more than a century. As early as the 1920s, the influence of histologic “grade,” a numeric expression of differentiation, on patient outcome was first analyzed. Well-differentiated cancer cells are associated with increased survival, whereas the presence of poorly differentiated cancer cells is predictive of poor clinical outcome. The fundamental conclusion that poorly differentiated tumors pursue a more aggressive course than their well differentiated counterparts has been repeatedly upheld in subsequent years.

Tumor grades are often presented on a scale of 1 to 3 or 1 to 4 where grade 1 represents cancers having well-differentiated, slowly dividing cells; grade 2 represents cancers having moderately differentiated cells; grade 3 represents cancers having poorly differentiated, rapidly dividing cells; and grade 4 represents cancers having undifferentiated cells. Cancer prognosis decreases with increasing grade.

Broders' method of grading, originally developed for squamous cell carcinoma, is still in use today. By this method, tumors are assigned one of four grades according to the percentage of tumor showing incomplete differentiation. Broders, JAMA 656-654 (1920); Broders, Ann. Surg. 73:141-60 (1921); and Broders Arch. Pathol Labl Med. 2:376-81 (1926). Low-grade, well-differentiated, tumors more closely resemble their benign counterpart, whereas the higher-grade, more poorly differentiated tumors have little resemblance. Formal grading systems have improved in recent years with stricter standardization of criteria. Elston, Aust NZ J. Surg. 54:11-15 (1984).

While grading has proved to be of great value in predicting cancer prognosis, it has not been appreciated that the grading of cancer cell differentiation may be applied to assess prospectively the clinical outcome of patients undergoing immunotherapeutic treatment regimens. Thus, there remains a need in the art for methods that may be used to predict therapeutic outcome of immunotherapeutic-based cancer treatment regimens.

### SUMMARY OF THE INVENTION

The present invention addresses these and other related needs by providing immunotherapeutic compositions and methods for inhibiting the growth of cancer cells in a patient having a moderately to well-differentiated grade of cancer. Also provided are methods for assessing in a cancer patient the susceptibility of cancer cells to immunotherapeutic compositions. Each of the immunotherapeutic compositions and methods presented herein is based upon the observation that the “grade” of a cancer cell, being a measure of the cell's differentiation state, is predictive of clinical outcome in cancer patients undergoing an immunotherapeutic treatment regimen.

Within certain embodiments, the present invention provides immunotherapeutic compositions comprising activated, isolated antigen presenting cells (APCs) wherein the APCs are obtained from a patient having a moderately to well differentiated grade of cancer. The APCs are stimulated by exposure ex vivo to a tumor-associated antigen (TAA). The tumor-associated antigen may be a tumor-specific antigen. The tumor-associated antigen and/or tumor-specific antigen may be a component of a protein conjugate wherein the protein conjugate comprises an N-terminal moiety and a C-terminal moiety. The APCs stimulated according to the present invention are effective in activating T-cells to produce a cytotoxic cellular response against either the N-terminal moiety or the C-terminal moiety. The level of T-cell activation is higher than that produced by the APCs when exposed to either the N-terminal moiety or the C-terminal moiety alone. Specific preferred embodiments provide that the APCs are dendritic cells (DCs).

Immunotherapeutic compositions of the present invention are particularly suited to the treatment of cancers such as, for example, soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate. Other cancers may also be treated. Exemplified herein are immunotherapeutic compositions for the treatment of prostate cancer.

In those embodiments where the cancer is prostate cancer, the differentiation state of the cancer cells may, for example, be determined by the Gleason score. Immunotherapeutic compositions may comprise APCs isolated from patients diagnosed with prostate cancers having a Gleason score of ≦7, wherein a Gleason score of ≦7 indicates the presence of moderately to well-differentiated cancer cells. Within certain embodiments, APCs are isolated from prostate cancer patients that are refractory to hormone ablation therapy. Other embodiments provide that the APCs are isolated from prostate cancer patients that are not refractory to hormone ablation therapy.

Certain aspects of the present invention provide immunotherapeutic compositions wherein the APCs are stimulated by a protein conjugate that is a fusion protein. According to these aspects of the invention, the fusion protein comprises an N-terminal moiety and a C-terminal moiety and may, additionally, include a linker peptide of one or more amino acids. Either of the N-terminal or C-terminal moieties may comprise a sequence having at least 70%, 80%, 90%, 95%, or 98% sequence identity with the sequence depicted in SEQ ID NO: 1 (huPAP) or may comprise an active fragment, derivative or variant of huPAP. Other embodiments provide immunotherapeutic compositions wherein the N-terminal moiety or the C-terminal moiety has the sequence depicted in SEQ ID NO: 1.

In other embodiments, the immunotherapeutic compositions of the present invention comprise APCs stimulated with a protein conjugate having a C-terminal moiety or an N-terminal moiety that is at least 70%, 80%, 90%, 95%, or 98% identical to the sequence depicted in SEQ ID NO: 3 (huGM-CSF) or an active fragment, derivative, or variant of huGM-CSF. Other embodiments provide immunotherapeutic compositions wherein the C-terminal moiety or the N-terminal moiety comprises the sequence depicted in SEQ ID NO: 3.

More preferred are immunotherapeutic compositions wherein the APCs are stimulated with a protein conjugate comprising an N-terminal moiety having at least 70%, 80%, 90%, 95%, or 98% sequence identity with the sequence depicted in SEQ ID NO: 1 (huPAP) or an active fragment, derivative, or variant of huPAP and a C-terminal moiety having at least 70%, 80%, 90%, 95%, or 98% sequence identity with the sequence depicted in SEQ ID NO: 3 (huGM-CSF) or an active fragment, derivative, or variant of huGM-CSF. Most preferred are immunotherapeutic compositions comprising APCs obtained from patients having moderately to well-differentiated cancer cells which APCs are stimulated with a protein conjugate comprising the sequence depicted in SEQ ID NO: 5.

The present invention is also directed to methods of inhibiting the growth of cancer cells in a patient having a moderately to well-differentiated grade of cancer. Within one embodiment, the methods comprise the steps of (a) determining in the patient the presence of moderately to well-differentiated cancer cells wherein moderately to well-differentiated cancer cells indicate a patient susceptible to treatment with an immunotherapeutic composition; and (b) administering to the patient with moderately to well-differentiated cancer cells a therapeutically effective dose of an immunotherapeutic composition. By these methods, a reduction in the progression of said cancer by 10%, 25%, or 50% indicates an inhibition of the growth of the cancer cells.

Within certain preferred embodiments, the present invention provides methods for inhibiting the growth of cancer cells in a patient having a moderately to well-differentiated grade of cancer which methods employ one of the immunotherapeutic compositions described above.

Alternative related embodiments provide methods for inhibiting growth of a cancer cell in a patient having moderately to well-differentiated cancer cells which methods comprise the steps of (a) determining in the patient the grade of the cancer cell wherein a moderately to well-differentiated cancer grade indicates a patient susceptible to treatment with an immunotherapeutic composition; (b) isolating antigen presenting cells (APCs) from the patient having a moderately to well-differentiated grade of cancer; (c) stimulating the APCs by exposure ex vivo to a protein conjugate comprising an N-terminal moiety and a C-terminal moiety, wherein the APCs are effective in activating T-cells to produce a cytotoxic cellular response against either the N-terminal moiety or the C-terminal moiety and wherein the level of the T-cell activation is higher than that produced by the APCs when exposed to the N-terminal moiety or to the C-terminal moiety alone; and (d) administering to the patient a therapeutically effective dose of the stimulated APCs. By these methods, a reduction in the progression of said cancer by 10%, 25%, or 50% indicates an inhibition of the growth of said cancer cells.

Within certain aspects of these methods, the cancer is selected from the group consisting of soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate. Other cancers may also be treated by the methods of the present invention. Preferred methods provide that the cancer is prostate cancer. In those embodiments wherein the cancer is prostate cancer, the cancer grade may, for example, be determined by Gleason score wherein a Gleason score of ≦7 indicates a patient susceptible to a treatment regimen employing an immunotherapeutic composition.

The present invention is also directed to methods for assessing in a cancer patient the susceptibility of cancer cells to treatment with an immunotherapeutic composition. Exemplary methods comprise the steps of (a) isolating from the patient a sample containing a cancer cell and (b) determining the differentiation and/or growth rate characteristics of the cancer cell, wherein the presence of a moderately to well-differentiated cancer cell indicates the susceptibility of the cancer cell to treatment with an immunotherapeutic composition.

The above-mentioned and additional features of the present invention and the manner of obtaining them will become apparent, and the invention will be best understood by reference to the following more detailed description, read in conjunction with the accompanying drawings. All references disclosed herein are hereby incorporated by reference in their entirety as if each was incorporated individually.

### BRIEF DESCRIPTION OF THE DRAWINGS AND SEQUENCE IDENTIFIERS

FIG. 1 is a graph depicting the time to disease progression (Kaplan-Meier method) for all hormone refractory prostate tumor patients treated with APCs either stimulated with a prostatic acid phosphatase (PAP)/granulocyte-macrophage colony stimulating factor (GM-CSF) fusion protein (APC8015) or unstimulated (Placebo) prior to administration.

FIG. 2 is a graph depicting the time to disease progression (Kaplan-Meier method) for all hormone refractory prostate tumor patients having a Gleason score of ≧8 treated with APCs either stimulated with a prostatic acid phosphatase (PAP)/granulocyte-macrophage colony stimulating factor (GM-CSF) fusion protein (APC8015) or unstimulated (Placebo) prior to administration.

FIG. 3 is a graph depicting the time to disease progression (Kaplan-Meier method) for all hormone refractory prostate tumor patients having a Gleason score of ≦7 treated with APCs either stimulated with a prostatic acid phosphatase (PAP)/granulocyte-macrophage colony stimulating factor (GM-CSF) fusion protein (APC8015) or unstimulated (Placebo) prior to administration.

FIG. 4 is a bar graph depicting the effect of PAP/GM-CSF on the proliferation of T-cells in a population of peripheral blood mononuclear cells.

FIG. 5 is a bar graph presenting data demonstrating that APC8015 induces a significant T-cell mediated immune response in prostate cancer patients as compared to an equivalent patient population receiving placebo.

FIG. 6 is a bar graph presenting data demonstrating that APC8015 induces a significant T-cell mediated immune response in prostate cancer patients having a Gleason score of ≦7 as compared to prostate cancer patients having a Gleason score of ≧8.

FIG. 7 is a graph depicting the time to onset of disease-related pain (Kaplan-Meier method) for prostate cancer patients having a Gleason score of ≦7 treated with APC8015 or placebo vs. prostate cancer patients having a Gleason score of ≧8 treated with APC8015 or placebo.

SEQ ID NO: 1 is the amino acid sequence of human prostatic acid phosphatase (huPAP) as encoded by the cDNA sequence depicted in SEQ ID NO: 2.

SEQ ID NO: 2 is the nucleotide sequence of a cDNA encoding human prostatic acid phosphatase (huPAP) as depicted in SEQ ID NO: 1.

SEQ ID NO: 3 is the amino acid sequence of human granulocyte-macrophage colony stimulating factor (huGM-CSF) as encoded by the cDNA sequence depicted in SEQ ID NO: 4.

SEQ ID NO: 4 is the nucleotide sequence of a cDNA encoding human granulocyte-macrophage colony stimulating factor (huGM-CSF) as depicted in SEQ ID NO: 3.

SEQ ID NO: 5 is the amino acid sequence of a human prostatic acid phosphatase/human granulocyte-macrophage colony stimulating factor (huPAP/huGM-CSF) fusion protein as encoded by the cDNA sequence depicted in SEQ ID NO: 6.

SEQ ID NO: 6 is the nucleotide sequence of a cDNA encoding a human prostatic acid phosphatase/human granulocyte-macrophage colony stimulating factor (huPAP/huGM-CSF) fusion protein as depicted in SEQ ID NO: 5.

### DETAILED DESCRIPTION OF THE INVENTION

As indicated above, the present invention provides immunotherapeutic compositions and methods for inhibiting the growth of cancer cells in a patient having a moderately to well-differentiated grade of cancer. Also provided are methods for assessing in a cancer patient the susceptibility of cancer cells to immunotherapeutic compositions. Each of the immunotherapeutic compositions and methods presented herein is based upon the observation that the “grade” of a cancer cell, being a measure of the cell's differentiation state, is predictive of clinical outcome in cancer patients undergoing an immunotherapeutic treatment regimen. Whereas poorly differentiated cells were found to be refractory to an immunotherapeutic treatment regimen, moderately to well-differentiated cells were highly susceptible to treatment with immunotherapeutic compositions.

As used herein, the term “differentiated” describes the extent to which cancer cells resemble the appearance of normal cells of the same tissue type. The degree of differentiation often relates to the clinical behavior, and hence prognosis, of a particular cancer. The differentiation state of cancer cells is commonly assessed through histological grading methodologies. The World Health Organization and American Joint Commission on Cancer have independently proposed comparable four grade systems for assessment of cancer cell differentiation based on histological parameters: Cells of grade 1 (G1) cancers are characterized as well-differentiated, slow growing cells that form low-grade tumors; grade 1 cancers are the least aggressive in behavior. Grade 2 (G2) cancer cells are moderately well-differentiated and form tumors that are intermediate in aggressive behavior. Conversely, the cells of grade 3 (G3) or grade 4 (G4) cancers are poorly differentiated or undifferentiated, respectively, divide rapidly and form high-grade tumors that are the most aggressive in behavior.

Although histologic grade is frequently utilized as a prognostic indicator for estimating the future course and outcome of disease—in particular for cancers such as soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate, it has not been previously recognized that histologic grade may be used as a reliable indicator of the efficacy of immunotherapeutic treatment regimens. Carriaga et al., Cancer Supp. 75(1):406-421 (1994). As part of the present invention, it was observed that well-differentiated (G1) and moderately differentiated (G2) cancer cells are susceptible to immunotherapeutic treatment regimens, whereas poorly differentiated (G3) or undifferentiated (G4) cells are refractory to treatment with immunotherapeutic compositions.

In one embodiment is provided the application of the Gleason score for assessing the histopathological grade of prostate cancer cells and for determining prospectively the clinical outcome of prostate cancer patients that are treated with an immunotherapeutic treatment regimen. Over 95% of prostate cancers are adenocarcinomas that arise from prostatic epithelial cells. Other rare histologies have been described, including mucinous or signet ring tumors, adenoid cystic carcinomas, carcinoid, large prostatic duct carcinomas (including the endometrial type) adenocarcinomas, and small cell undifferentiated cancers. Many studies have confirmed the primary prognostic importance of the degree of histologic differentiation of prostate adenocarcinoma. The degree of differentiation is typically graded by patterns of gland formation and, less importantly, by cytologic detail. The most widely accepted grading scheme for adenocarcinoma of the prostate is that developed by Gleason. Cancer Chemother. Rep. 50:125-128 (1966), incorporated by reference herein in its entirety. Gleason's original work demonstrated an association between a higher Gleason score and higher mortality, which others have confirmed; the Gleason score remains the most broadly applicable and prognostically useful histologic grading system. Gleason et al., J. Urol. 111:58-64 (1974); Gleason, Natl. Cancer Inst. Monograph 7:15 (1988); and Bostwick, C A Cancer J. Clin. 47:297-319 (1997).

Gleason's system for classifying prostate tumors is based on two levels of scoring, recognizing the heterogeneous differentiation in prostate carcinomas. The primary pattern of differentiation is assigned a Gleason grade of 1 to 5 based on the dominant morphology of the specimen and its departure from normal appearance; the secondary pattern (i.e. the next most common pattern) is also assigned a grade from 1-5 to achieve scores ranging from 2 to 10 based on patterns of tissue architecture. Lower Gleason scores (i.e. 2-4) describe well-differentiated, less aggressive cancer cells; intermediate Gleason scores (i.e. 5-7) are classified as moderately differentiated cancer cells; and higher scores (i.e. 8-10) describe poorly differentiated, aggressive tumors. As part of the present invention, it was recognized that prostate cancer cells having Gleason scores of ≦7, i.e. moderately to well-differentiated cancer cells, are generally susceptible to treatment with an immunotherapeutic treatment regimen, whereas cancer cells exhibiting a Gleason score of ≧8 are generally refractory to such treatment modalities.

In addition to the Gleason system for assessing histopathological parameters for prostate adenocarcinomas, numerous other analogous histological grading methodologies exist for achieving the differentiation state of a wide variety of cancer types. Methods for grading a wide range of cancers are well known in the art and are routinely employed for generating prognostic assessments. It will be appreciated that the present invention is not limited to the exemplary descriptions of cancer grading methodologies presented herein. Rather, the immunotherapeutic methods of use as well as methods for assessing the susceptibility of a cancer cell to immunotherapeutic treatment regimens disclosed herein may be broadly employed to treat and assess cancers characterized by moderately to well-differentiated grades of cancer.

Most cancers arising in the bladder are transitional cell carcinomas (TCCs). Some TCCs show a mixed pattern with squamous features or a glandular component. Martin et al., J. Clin. Pathol. 42:250-253 (1989). Less common pathologies are adenocarcinoma, squamous cell carcinoma, and small-cell carcinoma, which comprise approximately 6, 2, and less that 1% of bladder tumors, respectively. Tumor grading (typically grades I-III) is based on the number of mitoses, presence of nuclear abnormalities, and cellular atypia. A significant correlation exists between grade and prognosis. Heney et al., J. Urol. 130:1083-1086 (1983).

The International Federation of Gynecology and Obstetrics has adopted a methodology for grading endometrial cancers (FIGO grading system). Mikuta, Cancer 71:1460-3 (1993); and Silverberg et al., Armed Forces Institute of Pathology, pp 48-55 (Washington, D.C., Third Series, Fascicle 3, 1992). Within classic endometrioid adenocarcinomas, tumor grade is highly significant as an independent prognostic factor; less-differentiated tumors are associated with other poor prognostic factors, including deep myometrial penetration, vascular space invasion, and increasing staging. Morrow et al., Gynecol. Oncol. 40:55-65 (1991); Aalders et al., Obstet. Gynecol. 56:419-427 (1980); Chambers et al., Gynecol. Oncol. 27:180-188 (1987); Sutton et al., Am J. Obstet Gynecol 160:1385-1391 (1989); and Wharton et al., Surg. Gynecol. Obstet. 162:515-520 (1986). Carcinomas of the corpus are grouped with regard to the degree of differentiation of the adenocarcinoma as follows: G1 grade adenocarcinomas are well-differentiated and characterized by 5% or less of a nonsquamous or nonmorular solid-growth pattern; G2 grade adenocarcinomas are moderately differentiated and characterized by 6-50% of a nonsquamous or nonmorular solid-growth pattern; and G3 grade adenocarcinomas are poorly differentiated and characterized by more than 50% of a nonsquamous or nonmorular solid-growth pattern.

Several methodologies have been adopted for the grading of ovarian carcinomas. Shimizu et al., Gynecologic Oncology 70:2-12 (1998). Typically, grading may be achieved through assessment of cellular architecture, nuclear polymorphism, and mitotic count. Through architectural grading, the proportions of glandular, papillary, and solid growth are assessed; when more than 50% of the tumor is architecturally glandular, papillary, or solid, a grade of 1, 2, or 3 is assigned, respectively. Nuclear polymorphism is determined by measuring the variation in nuclear size and shape, chromatin texture, the nuclear:cytoplasmic ratio, and the number and size of nucleoli. Grade 1 indicates relative uniformity in vesicular nuclei, low nuclear:cytoplasmic ratio, and the absence of chromatin clumping or prominent nucleoli; grade 2 is assigned for nuclear size between 2:1 and 4:1, variation in shape, small but recognizable nucleoli, some chromatin clumping, and an absence of bizarre cells; grade 3 indicates a marked variation in nuclear size (greater than 4:1) and shape, high nuclear:cytoplasmic ratio, prominent chromatin clumping, thick nuclear membranes, large eosinophilic nucleoli; and the presence of bizarre cells. Mitotic count focuses on the presence of mitotic figures (MF) where nuclei with definite morphologic features of metaphase, anaphase, or telophase are counted in high-power microscopic fields (HPF). Up to 9 MF/10 HPF are assigned grade 1, 10-24 MF/10 HPF are assigned grade 2, while 25 or more MF/10 HPF are grade 3. Alternatively, FIGO grading, as derived from endometrial adenocarcinoma (see above), may also be employed as adopted by the Pathology Committee of the Gynecologic Oncology Group (GOG). Benda et al., GOG Pathology Manual (Buffalo, 1994). FIGO grading is based on the ratio of glandular or papillary structures versus solid tumor growth (Grade 1, <5% solid tumor; Grade 2, 6-50% solid tumor; and Grade 3, >50% solid tumor).

Breast cancers have been categorized into three histologic grades of malignancy, depending on the degree of gland and tubular formation, size of cells, size and differentiation of nuclei (nuclear pleomorphism), degree of hyperchromatism, and mitotic activity. Bloom et al., Br. J. Cancer 11:359 (1957) and Scharf et al., Lancet 2:582 (1938). Histologic grade 1 breast cancers are recognized as well-differentiated, grade 2 as moderately differentiated, and grade 3 as poorly differentiated. More specifically, when definite tubule formation is seen in at least 75% of the tumor area, a score of 1 is given; when less than 10% of the tumor shows definite tubule formation, a score of 3 is assigned; whereas a score of 2 is given to the intermediate category. In addition, nuclear pleomorphism and/or mitotic rate may be assessed to assign a histologic grade to breast cancers. If there is little variation and the nuclei appear quite regular, a score of 1 may be assigned while the presence of multiple nucleoli favor a score of 3. Breast cancers characterized by fewer than 10 mitoses per 10 high-power fields are assigned a score of 1 while more than 20 mitoses per 10 high-power fields indicates a score of 3. These three scores are combined to yield a grade. Grade 1 cancers have a combined score of 3 (the lowest possible score), 4 or 5; grade 2 is assigned for a combined score of 6 or 7; whereas grade 3 (high combined histologic grade) is given for cases that score 8 or 9 total points.

Soft tissue sarcomas are generally graded according to the methodology proposed by the National Cancer Institute. Costa et al., Cancer 53:530-41 (1984). In general, the more pleomorphic, more cellular, less differentiated tumors have the worse prognosis. Among the parameters measured to assess the cancer grade are the number of mitoses, the presence of myxoid areas, the extent of necrosis, and the differentiation of the tumor. Alternatively, the EORTC grading system may be employed to derive the cancer grade of soft tissue sarcoma. By the EORTC system, mitotic count and necrosis values are related by the following formula: Score=(0.732×necrosis)+(0.873×number of mitoses). Van Unnik, Hematology/Oncology Clin. of North America 9(3):677-700 (1995); and Coindre et al., Eur. J. Cancer 29A:2089-2093 (1993). By this system, grades of I through IV are derived wherein grades I and II correspond, respectively, to well- and moderately differentiated cancer cells; grade III corresponds to poorly differentiated cancer cells; and grade IV corresponds to undifferentiated cancer cells.

Bone sarcomas are generally classified according to the scheme proposed by the Mayo Clinic which relies upon cytologic features or products of the lesional cells. Dahlin et al., Bone Tumors: General Aspects and Data on 8542 Cases, ed. 4. (Springfield, Ill., Charles C. Thomas, 1986). Osteosarcomas, fibrosarcomas, and malignant fibrous histiocytomas are graded on a scale of 1 to 4. Chondrosarcoma and malignant vascular tumors are graded on a scale of 1 to 3. Osteosarcomas are generally high-grade tumors, having grades of 3 or 4, and, as such, may not be amenable to treatment regimens employing immunotherapeutics according to the present invention. Grade 1 and 2 osteosarcomas that are suitable for immunotherapeutic treatment regimens are rare. Such cancers are characterized by a slight degree of hypocellular and cytologic anaplasia. Chondrosarcomas are graded based primarily on cellularity, nuclear size, and hyperchromasia. Additional measurements that have been used are mitotic rate and proportion of multinucleate tumor cells (two or more nuclei within one lacuna). Grade 1, well-differentiated, cancers contain chondrocytes with a slight to moderate increase in nuclear size and variation in shape. Occasional multinucleated cells are present, and mitotic activity in generally absent. Chondroid matrix is abundant and necrosis is scant. Grade 2, moderately differentiated, lesions are more cellular and contain a greater degree of nuclear atypia and hyperchromasia as compared to grade 1 tumors. Multinucleation is more frequent, and occasional mitotic figures may be found. These tumors also contain less chondroid matrix than grade 1 lesions. Myxoid stromal changes and necrosis are commonly seen. Grade 3, poorly differentiated, cancers are highly cellular (especially at the periphery of tumor lobes), pleomorphic and exhibit easily identified nuclear anaplasia. The vast majority of chondrosarcomas are grade 1 or 2. See, Inwards et al., Hematology/Oncology Clin. North America, 9(3):545-569 (1995).

The malignancy scale of the WHO classification is the most generally accepted for histological grading of astrocytomas. Kleihues et al., Brain Pathol. 3:255-268 (1993); Kleihues et al., Histological Typing of Tumours of the Central Nervous System: World Health Organization International Histological Classification of Tumours. (Springer, Berlin, 1993); Daumas-Duport et al., Cancer 62:2152-65 (1988); and Kim et al., J. Neurosurg. 74:27-37 (1991). Grade 1, well-differentiated astrocytomas have bipolar, “piloid” cells, Rosenthal fibers, and eosinophilic granular bodies; grade 2 moderately differentiated astrocytomas are characterized by neoplastic fibrillar or gemistocystic astrocytes and nuclear atypia; grade 3, poorly differentiated astrocytomas add to the characteristics of grade 2 astrocytomas the presence of mitotic activity; and grade 4, undifferentiated astrocytomas exhibiT-cellular anaplasia, nuclear atypia, mitoses, vascular proliferation, and necrosis.

Immunotherapeutic Compositions

Within certain embodiments of the present invention are provided immunotherapeutic compositions for the treatment of cancers that are characterized by moderately to well-differentiated cancer cells such as those exhibiting a grade of 1 or 2 as defined above. Within certain embodiments, immunotherapeutic compositions exemplified herein comprise activated, isolated antigen presenting cells (APCs) obtained from patients diagnosed with a moderately to well-differentiated grade of cancer. APCs are stimulated by exposure ex vivo to a protein conjugate comprising an N-terminal moiety and a C-terminal moiety such that the APCs are effective in activating T-cells to produce a cytotoxic cellular response against either the N-terminal moiety or the C-terminal moiety. The level of T-cell activation achieved by immunotherapeutic compositions is higher than the level produced by APCs exposed singularly to either the N-terminal moiety or the C-terminal moiety alone.

(a) APCs and DCs

As used herein, the term “antigen presenting cells” or “APCs” refers to cells that are capable of activating T-cells, and include, but are not limited to, certain macrophages, B cells, and, most preferably, dendritic cells (DCs). “Potent antigen presenting cells” are cells that, after being pulsed with an antigen, can activate naïve CD8+ cytotoxic T-lymphocytes (CTL) in a primary immune response. “Dendritic cells” or “DCs” are members of a diverse population of morphologically similar cell types found in lymphoid or non-lymphoid tissues. These cells are characterized by their distinctive morphology and high levels of surface MHC-class II expression. Steinman et al., Ann. Rev. Immunol. 9:271 (1991), incorporated herein by reference. APCs and DCs can be isolated from a number of tissue sources, and conveniently, from peripheral blood, as described herein. Preferred immunotherapeutic compositions of the present invention employ APCs or DCs that are isolated from a cancer patient diagnosed with a moderately to well-differentiated grade of cancer.

APCs and DCs may be isolated by routine methodologies that are readily available in the art. An exemplary suitable methodology for isolation of DCs is disclosed in U.S. Pat. Nos. 5,976,546, 6,080,409, and 6,210,662, each of these patents in incorporated herein by reference. Briefly, buffy coats may be prepared from peripheral blood. Cells may be harvested from leukopacs, layered over columns of organosilanized colloidal silica (OCS) separation medium (prepared as described by Dorn in U.S. Pat. No. 4,927,749, incorporated herein by reference) at a density 1.0770 g/ml, pH 7.4, 280 mOsm/kg H₂O) in centrifuge tubes or devices. The OCS medium is preferably prepared by reacting and thus blocking the silanol groups of colloidal silica (approximately 10-20 nm diameter particles) with an alkyl trimethoxy silane reagent.

In one embodiment, the OCS density gradient material is diluted to an appropriate specific density in a physiological salt solution supplemented with polyvinylpyrrolidone (PVP) such as PVP-10 available from Sigma Chemical Co. (St. Louis, Mo.). The tubes are centrifuged and the peripheral blood mononuclear cells (PBMC), present at the interface, are harvested.

PBMC are resuspended and centrifuged again to remove platelets and may optionally be spun through columns of OCS (density 1.0650 g/ml, 280 mOsm/kg H₂O). The resulting interface and pelleT-cells are harvested and washed with D-PBS by centrifugation. The pellet fraction is resuspended in cell culture medium and cultured in a humidified 5% CO₂ incubator for 40 hours. Following incubation, the non-adherent T-cells are harvested. The purity of dendritic cells in the interface fraction may be quantified by FACS analysis.

The morphology of the cells can be evaluated using photomicroscopy. The DC enriched fraction contains large sized veiled cells with cytoplasmic processes extending from the cell surface, features characteristic of DC.

(b) Protein Conjugates

As indicated above, exemplary immunotherapeutic compositions according to the present invention may comprise APCs or DCs that have been stimulated ex vivo with a protein conjugate. Preferred protein conjugates comprise an N-terminal moiety and a C-terminal moiety wherein the N-terminal moiety includes at least a portion of a “tumor-associated antigen (TAA)” or an “oncogene product” and the C-terminal moiety includes at least a portion of an “antigen presenting cell binding protein” or, more preferably, a “dendritic cell binding protein.” Equally preferred are protein conjugates wherein the C-terminal moiety includes at least a portion of a “tumor-associated antigen” or an “oncogene product” and the N-terminal moiety includes at least a portion of an “antigen presenting cell binding protein” or a “dendritic cell binding protein.”

As used herein, the term “tumor-associated antigen” refers to an antigen that is characteristic of a tissue type, including specific tumor tissues. An example of a tumor-associated antigen expressed by a tumor tissue is the antigen prostatic acid phosphatase, which is present in over 90% of all prostate tumors. The term “oncogene product” refers to any protein encoded by a gene associated with cellular transformation. Examples of oncogene products include, for example, Her2, p21RAS, and p53.

The terms “antigen presenting cell binding protein” and “dendritic cell binding protein” refer to any protein for which receptors are expressed on an APC or a DC, respectively. Examples of APC binding proteins and DC binding proteins include, but are not limited to, GM-CSF, IL-1, TNF, IL-4, CD40L, CTLA4, CD28, and FLT-3 ligand.

“Protein conjugates,” as disclosed herein, refer to covalent complexes formed between the N-terminal moiety and the C-terminal moiety. Protein conjugates between tumor-associated antigens/tumor-specific antigens/oncogene products and antigen presenting cell binding proteins/dendritic cell binding proteins may be complexed either chemically or as a fusion protein as discussed in greater detail herein below.

Immunotherapeutic compositions exemplified herein comprise activated, isolated antigen presenting cells (APCs) obtained from patients diagnosed with moderately to well-differentiated grades of prostate cancer. The APCs were stimulated by exposure ex vivo to a protein conjugate comprising an N-terminal moiety including a portion of the prostate tumor-associated protein human prostatic acid phosphatase (huPAP) and a C-terminal moiety including a portion of the APC/DC binding protein human granulocyte-macrophage colony stimulating factor (huGM-CSF). APCs stimulated in this fashion were effective in activating T-cells to produce a cytotoxic cellular response against the N-terminal PAP moiety. The level of T-cell activation achieved by this exemplary immunotherapeutic composition was higher than that produced by APCs exposed exclusively to PAP alone.

The exemplary PAP/GM-CSF protein conjugate disclosed herein was previously described within U.S. Pat. Nos. 5,976,546, 6,080,409, and 6,210,662 and is presented herein as SEQ ID NO: 5. Each of these patents in incorporated herein by reference. This protein conjugate is a fusion protein between a 386 amino acid portion of PAP at the N-terminus and a 127 amino acid portion of GM-CSF at the C-terminus. The complete amino acid sequences of huPAP and huGM-CSF are presented herein as SEQ ID N Os: 1 and 3, respectively. In addition, the PAP/GM-CSF fusion protein of SEQ ID NO: 5 further comprises, between the N-terminal moiety and the C-terminal moiety, a two amino acid peptide linker having the sequence gly-ser.

Equally suited to the practice of the present invention are PAP/GM-CSF protein conjugates, including fusion proteins, comprising sequence variations within the amino acid sequences of the PAP and/or GM-CSF moieties. For example, the present invention contemplates protein conjugates wherein the PAP and/or the GM-CSF moieties are at least 70% identical to the amino acid sequences recited in SEQ ID NOs: 1 and 3, respectively. More preferred are PAP and/or GM-CSF moieties that are at least 80%, 90%, 95% and 98% identical to the amino acid sequences recited in SEQ ID NOs: 1 and 3, respectively.

As pointed out above, protein complexes may be formed through chemical means, such as by conventional coupling techniques, or as fusion proteins generated by expression of DNA constructs. Methodologies for generating protein complexes, whether coupled chemically or in the form of fusion proteins, are well known and readily available in the art. For example, the N-terminal and C-terminal moieties can be coupled using a dehydrating agent such as dicyclohexylcarbodiimide (DCCI) to form a peptide bond between the two peptides. Alternatively, linkages may be formed through sulfhydryl groups, epsilon amino groups, carboxyl groups or other reactive groups present in the polypeptides, using commercially available reagents. (Pierce Co., Rockford, Ill.).

Conventional molecular biology and recombinant DNA techniques for generating fusion proteins are explained fully in the literature. See, e.g., Sambrook et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984). Each of these publications is incorporated herein by reference in its entirety.

Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences that may be usefully employed as linkers include those disclosed in Maratea et al., Gene 40:39-46 (1985); Murphy et al., Proc. Natl. Acad. Sci. USA 83:8258-8262 (1986); U.S. Pat. No. 4,935,233; and U.S. Pat. No. 4,751,180. The linker sequences are generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Protein complexes between the N-terminal and C-terminal moieties may be generated recombinantly as fusion proteins as exemplified herein by the prostatic acid phosphatase (PAP)/granulocyte-macrophage colony stimulating factor (GM-CSF) fusion protein that was generated by cloning huPAP from a prostate carcinoma cell line and huGM-CSF from a PBMNC library. The stop codon at the 3′ end of PAP coding region was removed by standard mutagenesis methodology and replaced with a Bam HI restriction endonuclease site to facilitate the in-frame fusion of DNA encoding PAP to DNA encoding GM-CSF and, thereby, generating a six-nucleotide region encoding the polypeptide linker gly-ser juxtaposed between the N-terminal PAP and C-terminal GM-CSF moieties.

As noted above in the context of the PAP/GM-CSF fusion protein exemplified herein, it will be appreciated that protein complexes according to the present invention may comprise variants of the N-terminal and/or the C-terminal moieties without adversely affecting the functional properties of the tumor-associated antigen (TAA), the oncogene product, or the antigen presenting/dendritic cell binding protein. A polypeptide or protein “variant,” as used herein, is a polypeptide or protein that differs from a native polypeptide or protein in one or more substitutions, deletions, additions and/or insertions, such that the functional activity of the polypeptide or protein is not substantially diminished. In other words, the ability of a variant to react with or be processed by an antigen presenting or dendritic cell may be enhanced or unchanged, relative to the native protein, or may be diminished by less than 50%, and preferably less than 20%, relative to the native protein, without affecting the efficacy of the resulting immunotherapeutic composition.

Such variants may generally be identified by modifying amino acid sequence of the N-terminal and/or C-terminal moiety and evaluating the reactivity of the modified polypeptide with antigen presenting/dendritic cells or with antisera raised against the native tumor-associated antigen (TAA) or oncogene product. Such modification and evaluation may be achieved through routine application of molecular and cell biology techniques that are well known in the art.

Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminus of the mature protein. Polypeptide variants preferably exhibit at least about 70%, more preferably at least about 80% or 90% and most preferably at least about 95% or 98% sequence identity to the native polypeptide or protein.

Preferably, variants contain “conservative amino acid substitutions” as defined as a substitution in which one amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

Variants may additionally, or alternatively, be modified by, for example, the deletion or addition of amino acids that have minimal influence on the immunogenicity, secondary structure and hydropathic nature of the polypeptide.

As noted above, polypeptides or proteins may comprise a signal (or leader) sequence at the N-terminal end of the protein which co-translationally or post-translationally directs transfer of the protein.

Polypeptides may be prepared using any of a variety of well known techniques. Recombinant polypeptides encoded by DNA sequences as described above may be readily prepared from the DNA sequences using any of a variety of expression vectors known to those of ordinary skill in the art. Expression may be achieved in any appropriate hosT-cell that has been transformed or transfected with an expression vector containing a DNA molecule that encodes a recombinant polypeptide. Suitable hosT-cells include prokaryotes, yeast and higher eukaryotic cells. Preferably, the hosT-cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146 (1963). Equipment for automated synthesis of polypeptides is commercially available from suppliers such as Perkin Elmer/Applied BioSystems Division (Foster City, Calif.), and may be operated according to the manufacturer's instructions.

Within certain specific embodiments, a polypeptide may be a fusion protein that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Alternative Immunotherapeutic Compositions

As indicated above, within certain embodiments, the present invention provides methods that employ one or more immunotherapeutic composition in the treatment of cancer patients wherein the cancer cells are moderately to well-differentiated. In addition to the immunotherapeutic compositions described above, these methods may employ other immunotherapeutic compositions that are readily available in the art or that may otherwise be prepared through routine experimentation.

Within certain embodiments, immunotherapeutic compositions may comprise active immunotherapeutics, in which treatment relies on the in vivo stimulation of the endogenous host immune system to react against tumors with the administration of immune response-modifying agents (such as polypeptides and polynucleotides as provided herein).

Within other embodiments, immunotherapeutic compositions may comprise passive immunotherapeutics, in which treatment involves the delivery of agents with established tumor-immune reactivity (such as effector cells or antibodies) that can directly or indirectly mediate antitumor effects and does not necessarily depend on an intact host immune system. Examples of effector cells include T-cells as discussed above, T lymphocytes (such as CD8+cytotoxic T lymphocytes and CD4+ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T-cell receptors and antibody receptors specific for the polypeptides recited herein may be cloned, expressed and transferred into other vectors or effector cells for adoptive immunotherapy.

The immunotherapeutic compositions described herein may be used to stimulate an immune response against cancer. Immunotherapeutic compositions may be administered either prior to or following surgical removal of primary tumors and/or treatment such as administration of radiotherapy or conventional chemotherapeutic drugs. Administration of the immunotherapeutic compositions may be by any suitable method, including administration by intravenous, intraperitoneal, intramuscular, subcutaneous, intranasal, intradermal, anal, vaginal, topical and oral routes.

Immunotherapeutic compositions that are suitable for use in these methods include, for example, immunotherapeutic polypeptides, immunotherapeutic antibodies, polynucleotide-based anti-cancer vaccines, cell-based immunotherapeutics and combination compositions comprising one or more polypeptide-, antibody-, polynucleotide-, and/or cell-based immunotherapeutic. Each of the immunotherapeutic compositions presented herein share one or more of the properties of amplifying an immune response and/or breaking antigen-specific tolerance.

(a) Immunotherapeutic Polypeptides

In addition to the protein complexes described herein above, the present invention also contemplates the use of immunotherapeutic polypeptides for the treatment of cancers characterized by moderately to well differentiated cancer cells. Thus, polypeptides suitable for use in the present methods include immunogenic polypeptides, wherein immunogenic is defined as the capacity of the polypeptide to react detectably within an immunoassay, such as an ELISA and/or a T-cell stimulation assay, using antisera and/or T-cells isolated from a patient afflicted with cancer. Methodology for screening for immunogenic activity is well known to those of skill in the art. For example, exemplary screening methodology are disclosed within Harlow and Lane, Antibodies: A Laboratory Manual (Cold Spring Harbor Laboratory, 1988). Thus, a polypeptide may be immobilized on a solid support and contacted with patient sera to permit binding of antibodies within the sera to the immobilized polypeptide. Unbound sera may be removed and bound antibodies detected using, for example, Protein A carrying a detectable label.

Exemplary polypeptides suitable for use in the present invention include most typically tumor-associated and/or tumor-specific polypeptides such as polypeptides displaying an increased level of expression in tissue and/or tumor samples including, for example, samples isolated from patient with a cancer such as a soft tissue sarcoma, lymphoma, or cancer of the brain, esophagus, uterine cervix, bone, lung, endometrium, bladder, breast, larynx, colon/rectum, stomach, ovary, pancreas, adrenal gland, or prostate. Polypeptides identified as having increased expression in other cancers may also be suitably employed. Included are polypeptides that are expressed in a substantial portion of tumor samples, for example, greater than about 20%, or greater than about 30%, or more than about 50% or more of tumor samples tested, generally at a level that is at least two-fold, most commonly at least five-fold, greater than the level of expression in normal tissues.

It will be understood by those of skill in the art that the immunogenic portions of such tumor-associated and/or tumor-specific polypeptides may also be employed in the methods of the present invention. An “immunogenic portion” is defined herein as a fragment of an immunogenic polypeptide that is by itself immunologically reactive with B-cells and/or T-cell surface antigen receptors that specifically bind to the immunogenic polypeptide. Immunogenic portions may be identified using routine methodologies including those presented within Paul, Fundamental Immunology, 3^{rd} ed., 243-247 (Raven Press, 1993). Exemplary techniques include screening polypeptides for the ability to react with antigen-specific antibodies, antisera and/or T-cell lines or clones.

An immunogenic portion of a polypeptide includes those sequences that react with antisera and/or T-cells at a level that is not substantially less than the reactivity of the full-length polypeptide. Typically, the level of immunogenic activity of the immunogenic portion is at least about 50%, more typically at least about 70%, and most typically greater than about 90% of the immunogenicity for the full-length polypeptide.

Thus, immunotherapeutic polypeptides useful in the methods of the present invention are capable of eliciting T-cells and/or antibodies that are immunologically reactive with one or more tumor-specific and/or tumor-associated polypeptide as described above.

It will be apparent to one skilled in the art that tumor-specific and/or tumor-associated polypeptides may be recognized as “self” polypeptides by the immune system of a patient and, therefore, may be poor stimulators of a CD8+/CD4+ T-cell response. Thus, the present invention contemplates that xenogeneic polypeptides, especially those xenogeneic polypeptides that encompass the immunogenic portion of the tumor-associated and/or tissue-specific polypeptide may alternatively be used in the methods of the present invention. Use of such xenogeneic polypeptides may, therefore, be used to overcome immune tolerance to the particular self polypeptide. Exemplary xenogeneic polypeptides include polypeptides isolated from a mouse, rat, monkey, pig and/or other non-human animal.

As with the fusion proteins described above, polypeptides of the present invention may be prepared using any of a variety of well known synthetic and/or recombinant techniques readily available in the art. Polypeptides, portions, and other variants that are less than about 150 amino acids may be generated by synthetic means, for example, using any of the commercially available solid-phase techniques, such as the Merrifield solid-phase synthesis methodology as described above.

(b) Polynucleotides and Polynucleotide-based Therapeutics

Methods for the treatment of moderately to well-differentiated cancers, as presented herein, may employ one or more anti-cancer vaccine, the most common of which are the polynucleotide-based anti-cancer vaccines. Regardless of the specific features of a given anti-cancer vaccine, they all have in common the capacity to stimulate an anti-cancer immune response. The antigenic portion(s) of the vaccine may be delivered in the form of peptides, proteins, and fusion proteins, as disclosed herein above, and/or may be delivered in the form of a polynucleotide such as, for example, an RNA, a DNA and/or a virus such as adenovirus, adeno-associated virus, vaccinia virus or other virus known in the art.

Thus, the methods of the present invention may employ a polynucleotide, including a single-stranded or double-stranded polynucleotide, and may be DNA (such as, genomic, cDNA or synthetic DNA) or RNA (including HnRNA and mRNA). Suitable polynucleotides most typically comprise an endogenous sequence that encodes an immunogenic polypeptide, or portion thereof, including xenogeneic polypeptides and portions.

Polynucleotides encoding immunotherapeutic polypeptides may be combined with other DNA sequences, such as promoters, polyadenylation signals, additional restriction enzyme sites, multiple cloning sites, other coding segments, and the like, to enhance and/or facilitate expression of the polynucleotide encoded polypeptide.

Polynucleotides may be readily prepared by, for example, directly synthesizing the fragment by chemical means, as is commonly practiced using an automated oligonucleotide synthesizer. Alternatively, fragments may be obtained by application of nucleic acid reproduction technology, such as PCR™ technology of U.S. Pat. No. 4,683,202, by introducing selected sequences into recombinant vectors for recombinant production, and by other recombinant DNA techniques generally known to those of skill in the art. See, generally, Sambrook et al., Molecular Cloning: A Laboratory Manual, Cold Spring Harbor Laboratories (Cold Spring Harbor, N.Y., 1989).

Suitable polynucleotides that express immunotherapeutic polypeptides may be identified by, for example, screening a microarray of cDNAs for tumor-associated and/or tumor-specific expression (i.e. expression that is at least two-fold greater than in a distinct tissue and/or a normal tissue). Exemplary suitable microarray screening technology includes the technology of Affymetrix, Inc. (Santa Clara, Calif.) and may be employed according to the manufacturer's instructions. See, Schena et al., Proc. Natl. Acad. Sci. USA 93:10614-10619 (1996) and Heller et al., Proc. Natl. Acad. Sci. USA 94:2150-2155 (1997).

Expression of a desired polypeptide may be achieved by inserting a corresponding polynucleotide into an appropriate expression vector, i.e. a vector that contains the necessary elements for transcription and translation of the inserted coding sequence. Exemplary techniques for achieving expression of a polynucleotide encoding an immunotherapeutic polypeptide are presented within, for example, Sambrook et al., Molecular Cloning, A Laboratory Manual, supra, and Ausubel et al., Current Protocols in Molecular Biology, supra.

A variety of expression vector/hosT-cell systems may be utilized to express a tumor-associated and/or tumor-specific polynucleotide. Expression systems comprise microorganisms, such as bacteria transformed with recombinant bacteriophage, plasmid, or cosmid DNA expression vectors; yeast transformed with yeast expression vecots; insecT-cell systems infected with virus expression vectors; planT-cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

In addition to expressing an immunotherapeutic polypeptide of interest, polynucleotides encoding such polypeptides may be administered to a patient by utilizing any one of a variety of delivery systems known to those of skill in the art. Exemplary gene delivery techniques are described in Rolland, Crit. Rev. Therap. Drug Carrier Systems 15:143-198 (1998) and references cited therein. Suitable expression systems will contain DNA regulatory sequences (i.e. promoters and transcriptional termination signals) for expression in the patient. Most commonly, systems for expressing polynucleotides in a patient are viral-based systems. For example, retrovirus-, adenovirus-, adeno-associated virus-, pox virus-, vaccinia-, avipoxvirus-, and alphavirus-based systems have all been described.

Suitable retrovirus-based gene expression systems are described within U.S. Pat. No. 5,219,740; Miller et al., BioTechniques 7:980-990 (1989); Miller, Human Gene Therapy 1:5-14 (1990); Scarpa et al., Virology 180:849-852 (1991); Burns et al., Proc. Natl. Acad. Sci. USA 90:8033-8037 (1993); and Borris-Lawrie et al., Cur. Opin. Genet. Develop. 3:102-109 (1993).

Exemplary adenovirus-based systems are presented within Haj-Ahmad et al., J. Virol. 57:267-274 (1986); Bett et al., J. Virol. 67:5911-5921 (1993); Mittereder et al., Human Gene Therapy 5:717-729 (1994); Seth et al., J. Virol. 68-933-940 (1994); Barr et al., Gene Therapy 1:51-58 (1994); Berkner, BioTechniques 6:616-629 (1988); and Rich et al., Human Gene Therapy 4:461-476 (1993).

Adeno-associated virus (AAV)-based gene expression systems are disclosed in U.S. Pat. Nos. 5,173,414 and 5,139,941; Lebkowski et al., Molec. Cell. Biol. 8:3988-3996 (1988); Vincent et al., Vaccines 90 (Cold Spring Harbor Press, 1990); Carter, Current Opinions in Biotech. 3:533-539 (1992); Muzyczka, Current Topics in Microbiol. and Immunol. 158:97-129 (1992); Kotin, Human Gene Therapy 5:793-801 (1994); Shelling et al., Gene Therapy 1:165-169 (1994); and Zhou et al., J. Exp. Med. 179:1867-1875 (1994).

(c) Immunotherapeutic Antibodies

Immunotherapeutic antibodies typically are monoclonal antibodies directed against tumor-associated antigens. For example, similarly to inducing an immune response against PAP by means of vaccination with PAP/GM-CSF pulsed vaccines, PAP-directed immunity can also b e induced b y infusion of PAP-specific monoclonal antibodies. Such antibodies bind the antigen in vivo and direct APC towards it. After such induced invasion of APC into the tumor they induce subsequently tumor-specific immunity similar to a vaccine. Other targets for such tumor-specific antibodies are well known in the art. They include Her-2/neu, CEA, CD20, CEA, VEGF, and other tumor-associated antigens.

Thus, methods of the present invention may employ one or more antibody, or antigen-binding fragment thereof, wherein the antibody and/or fragment specifically binds to the antigen and, thereby, induces an immune response. More specifically, antibodies and/or antigen-binding fragments exhibit immunological binding to a tumor-associated and/or a tumor-specific antigen and/or to a xenogeneic variant thereof. An antibody, or antigen-binding fragment thereof, is said to “specifically bind” and/or be “immunologically reactive” to an immunotherapeutic polypeptide antigen if it reacts at a detectable level (by, for example, an ELISA assay) with the polypeptide, and does not react detectably with unrelated polypeptides, and does not react detectably with unrelated polypeptides under similar conditions.

Immunological binding refers to the non-covalent interactions between an antibody and an antigen for which the immunoglobulin is specific. The affinity of immunological binding interactions is generally expressed in terms of the dissociation constant (K_{d}) of the interaction, wherein a smaller K_{d} represents a greater affinity. Immunological binding properties of an antibody to its cognate polypeptide may be quantified using methods well known to those of skill in the art. One exemplary method entails measuring the rates of antigen-binding site/antigen complex formation and dissociation, wherein those rates depend on the concentrations of the complex partners, the affinity of the interaction, and on geometric parameters that equally influence the rate in both directions. Both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, Davies et al., Ann. Rev. Bioch. 59:439-473 (1990).

An “antigen-binding site,” or “binding portion” of an antibody refers to the part of the antibody that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” that are interposed between more conserved flanking stretches known as “framework regions” or “FRs.” The term “FR” refers to amino acid sequences that are naturally found between and adjacent to hypervariable regions (CDRs) in an immunoglobulin. In an antibody molecule, the three hypervariable regions of a light chain and the three hypervariable regions of a heavy chain are disposed relative to each other in three dimensional space to form an antigen binding surface. The antigen-binding surface is complementary to the three-dimensional surface of a bound antigen, and the three hypervariable regions of each of the heavy and light chains are referred to as “complementarity-determining regions” or “CDRs.”

Antibodies may be prepared by any of a variety of techniques known to those of skill in the art. See, Harlow and Lane, Antibodies: A Laboratory Manual, supra. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheeps, or goats). In this step, the immunogenic polypeptides, described above, may be used without further modification. Alternatively, a superior immune response may be elicited if the polypeptide is joined to a carrier protein, such as bovine serum albumin or keyhole limpet hemocyanin. The immunogenic polypeptide is injected into the animal host and the animals bled periodically. Polyclonal antibodies specific for the polypeptide may be purified from the antisera by, for example, affinity chromatography using a polypeptide coupled to a suitable solid support.

Monoclonal antibodies specific for an immunogenic polypeptide may be prepared, for example, using the technique of Kohler and Milstein, Eur. J. Immunol. 6:511-519 (1976). These methods involve the preparation of immortal cell lines capable of producing antibodies having the desired specificity (i.e. reactivity with the immunogenic polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. For example, the spleen cells and myeloma cells may be combined with a nonionic detergent for a few minutes and then plated at low density on a selective medium that supports the growth of hybrid cells, but not myeloma cells. A preferred selection technique uses hypoxanthine aminopterin thymidine (HAT) selection. Single colonies from the cell hybrids are selected and the culture supernatants tested for binding activity against the polypeptide. Hybridomas having high reactivity and specificity are preferred. Monoclonal antibodies may be isolated from the supernatants of growing hybridoma colonies and purified by affinity chromatography using the immunogenic polypeptide, or immunogenic portion thereof, used as the immunogen to generate the antibody.

A number of therapeutically useful molecules comprise antigen-binding sites that are capable of exhibiting the immunotherapeutic activity of the antibody molecule. The proteolytic enzyme papain preferentially cleaves IgG molecules to yield several fragments, two of which (the “F(ab)” fragments) each comprise a covalent heterodimer that includes an intact antigen binding site. The enzyme pepsin is able to cleave IgG molecules to provide several fragments, including the “F(ab′)₂” fragment that comprises both antigen-binding sites.

An “Fv” fragment can be produced by preferential proteolytic cleavage of an IgM, IgG, or IgA immunoglobulin molecule. Fv fragments are, however, more commonly derived using recombinant techniques known in the art. The Fv fragment includes a non-covalent V_{H}::V_{L} heterodimer including an antigen binding-site that retains much of the antigen recognition and binding capabilities of the native antibody. Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980).

A single chain Fv (“scFv”) polypeptide is a covalently linked V_{H}::V_{L} heterodimer that is expressed from a gene fusion including V_{H}- and V_{L}-encoding genes linked by a peptide-encoding linker. Huston et al., Proc. Nat. Acad. Sci. USA 85(16):5879-5883 (1988). A number of methods have been described to facilitate the generation of scFv molecules that will fold into a three-dimensional structure substantially similar to the structure of an antigen-binding site. U.S. Pat. Nos. 5,091,513, 5,132,405, and 4,946,778. Each of these patents is incorporated herein by reference.

In order to reduce the immune response directed against a non-human antibody when administered to a human, immunotherapeutic antibodies of the present invention also include “chimeric” and “humanized” monoclonal antibodies comprising variable domains and complementarity-determining regions (CDRs) from the antigen-binding site of a non-human immunoglobulin, respectively. Preparation of chimeric antibodies is presented within U.S. Pat. No. 4,816,567 to Cabilly while humanized antibodies are described within U.S. Pat. Nos. 5,530,101, 5,585,089, 5,693,762, and 6,180,370 to Queen; U.S. Pat. No. 5,859,205 to Adair; and U.S. Pat. No. 5,225,539 to Winter. Each of these patents is incorporated herein by reference.

Each antibody variable domain contains three hypervariable CDR regions (CDR1, CDR2, and CDR3) that are separated by framework regions (FR), which provide support to the CDRs and define the spatial relationship of the CDRs relative to each other. An antigen binding site includes six CDRs, three from the heavy chain variable region and three from the light chain variable region. Amino acid residues of CDRs contact the bound antigen, with the strongest contacts provided through the heavy chain CDR3.

There are four FR regions in each of the heavy and light chain variable domains. Some FR amino acid residues may contact bound antigen; however, FRs are primarily responsible for folding the V regions into the antigen-binding site. Within FRs, certain amino acid residues and certain structural features are very highly conserved. For example, all V region sequences contain an internal disulfide loop of around 90 amino acid residues. When the V regions fold into a binding-site, the CDRs are displayed as projecting loop motifs that form an antigen-binding surface. Conserved structural regions within the FRs influence the folded shape of the CDR loops to form certain “canonical” structures—regardless of the precise CDR amino acid sequence. And, certain FR residues participate in non-covalent interdomain contacts that stabilize the interaction of the antibody heavy and light chains.

Humanized and/or chimeric immunotherapeutic antibodies may be further modified through a process of “veneering” wherein amino acid residues within the FR regions are replaced with human FR residues in order to provide a xenogeneic molecule comprising an antigen-binding site that retains substantially all of the native FR polypeptide folding structure. Veneering techniques are based on the understanding that ligand binding characteristics of an antigen-binding site are determined primarily by the structure and relative disposition of the heavy and light chain CDRs within the antigen-binding surface. Davies et al., Ann. Rev. Biochem. 59:439-473 (1990). Thus, antigen binding specificity can be preserved in a humanized antibody only where the CDR structures, their interaction with each other, and their interaction with the rest of the V region domains are carefully maintained. By using veneering techniques, exterior (e.g., solvent-accessible) FR residues that are readily encountered by the immune system are selectively replaced with human residues to provide a hybrid molecule that comprises either a weakly immunogenic or substantially non-immunogenic veneered surface.

Within other embodiments, methods of the present invention may more suitably employ one or more fully-human immunotherapeutic antibody. For example, and as pointed out above, it is common for non-human antibodies, including humanized and chimeric antibodies, to elicit an anti-immunoglobulin immune response when administered in vivo to a human.

Methodology for generating fully-human antibodies are readily available in the art and is most frequently achieved through (1) immunization of a transgenic animal with an immunogenic polypeptide wherein the animal's antibody repertoire is replaced with a human antibody repertoire or (2) screening of a phage display antibody library with an immunogenic polypeptide and isolating the polynucleotide sequences encoding the human antibody heavy and light chains.

Transgenic animal systems suitable for generating immunotherapeutic antibodies for use in the methods of the present invention are disclosed within U.S. Pat. Nos. 6,150,584, 6,114,598, 6,162,963, 6,075,181, and 5,770,429. Phage display methodologies are presented within U.S. Pat. Nos. 6,248,516, 6,291,158, 6,291,159, 6,291,160, 6,291,161, 5,969,108, 6,172,197, 5,885,793, 6,265,150, 5,223,409, 5,403,484, 5,571,698, 5,837,500, and 6,300,064. Each of these patents is incorporated herein by reference.

Immunotherapeutic antibodies suitable for use in the methods of the present invention may additionally comprise one or more therapeutic agent such as, for example, a radioisotope, differentiation inducer, drug, toxin, and/or derivatives thereof. Exemplary radioisotopes include ⁹⁰Y, ¹²³I, ¹²⁵I, ¹³¹I, ¹⁸⁶Re, ²¹¹At, and ²¹²Bi. Suitable drugs include methotrexate and pyrimidine/purine analogs. Differentiation inducers include phorbol esters and butyric acid. And toxins include ricin, abrin, diphtheria toxin, cholera toxin, gelonin, pseudomonas exotoxin, Shigella toxin, and pokeweed antiviral protein.

Therapeutic agents may be coupled to an immunotherapeutic antibody either directly or indirectly (i.e. through a linker moiety). Methodologies for coupling therapeutic agents to antibodies are well known in the art. For example, U.S. Pat. No. 4,671,958 to Rodwell discloses suitable bifunctional and polyfunctional linker systems and methodology.

Cleavable linkers may be used alternatively when the toxicity of the un-coupled therapeutic agent exceeds its toxicity when coupled to the antibody. Cleavable linkers suitable for coupling therapeutic agents to antibodies for use in the methods of the present invention include, for example, linker groups that are cleavable by (1) reduction of a disulfide bond (U.S. Pat. No. 4,489,710); (2) irradiation of a photolabile bond (U.S. Pat. No. 4,625,014); (3) hydrolysis of derivatized amino acid side chains (U.S. Pat. No. 4,638,045); (4) serum complement-mediated hydrolysis (U.S. Pat. No. 4,671,958); and (5) acid-catalyzed hydrolysis (U.S. Pat. No. 4,569,789). Each of these patents is incorporated herein by reference.

(d) Cell-based-Immunotherapeutics

Cell-based immunotherapeutic compositions include antigen presenting cell (APC) and dendritic cell (DC) vaccines that have been prepared by methods other than described above. Alternatively, or additionally, cell-based compositions suitable for use in the methods of the present invention may include one or more T-cell population wherein the T-cells are specific for a tumor-associated and/or tumor-specific polypeptide as described herein above.

Exemplary such APC/DC preparations include but are not limited to APC and DC vaccines that have been prepared from cells that have been cultured in cytokines such as GM-CSF, IL-4 and TNF-alpha. Also, the APC or DC may have been exposed to tumor-specific antigens in form of either peptides, proteins, fusion proteins, nucleic acids such as RNA, DNA or viruses such as adenovirus, adeno-associated virus, vaccinia virus or other methods known in the art. Furthermore, tumor-infiltrating lymphocyte (TIL) cells having specificity for moderately to well-differentiated cancer cells may be used. TIL populations may be isolated from the cancer, grown ex vivo in the presence of IL-2 and re-administered to the cancer patient through standard adoptive transfer methodology. Suitable TIL populations contain mainly T lymphocytes, including both CD4+ and CD8+ T-cells.

T-cells specific for one or more polypeptide may be prepared in vitro or in vivo, using standard methodologies available to those of skill in the art. For example, T-cells may be isolated from bone marrow, peripheral blood, or a fraction of bone marrow or peripheral blood isolated from the cancer patient, using a commercially available cell separation system such as Isolex™ (Nexell Therapeutics, Inc., Irvine, Calif.) or those described within U.S. Pat. Nos. 5,240,856 and 5,215,926 and PCT Patent Application Nos. WO 89/06280, WO 91/16116, and WO 92/07243. Each of these patents is incorporated herein by reference.

T-cells may be stimulated with an immunotherapeutic polypeptide, a polynucleotide encoding such a polypeptide, and/or an antigen presenting cell (APC) or dendritic cell (DC) that presents at least a portion of the immunotherapeutic polypeptide. Such stimulation may be performed under conditions and for a time sufficient to permit the generation of T-cells that are specific for the immunotherapeutic polypeptide of interest.

T-cells are specific for an immunotherapeutic polypeptide if the T-cells specifically proliferate, secrete cytokines, and/or kill targeT-cells coated with the polypeptide or expressing a polynucleotide encoding the polypeptide. T-cell specificity may be evaluated using any of a number of methodologies known in the art such as, for example, a chromium release assay or a proliferation assay wherein a stimulation index of more than two-fold increase in lysis and/or proliferation, compared with negative controls, indicates T-cell specificity. Such assays may be performed, for example, as described in Chen et al., Cancer Res. 54:1065-1070 (1994).

Alternatively, detection of the proliferation of T-cells may be accomplished by, for example, measuring an increased rate of DNA synthesis (e.g., by the amount of tritiated thymidine incorporated into DNA). Contact with an immunotherapeutic polypeptide will typically result in at least a two-fold increase in proliferation of T-cells. Activation of the T-cells may be measured using a standard cytokine assay such as TNF or IFNγ release. See, e.g., Coligan et al., Current Protocols in Immunology, Vol. 1 (Wiley Interscience, 1998). Generally, T-cells suitable for immunotherapeutic purposes in the current methods are either CD4+ or CD8+ T-cells that proliferate in response to an immunotherapeutic polypeptide, polynucleotide, or APC/DC.

(e) Combination Immunotherapeutics

Within certain embodiments, the present invention also provides combined immunotherapeutic compositions comprising two or more immunotherapeutic agents or compositions as described herein above. Each of the individual immunotherapeutic agents may be administered individually or may be combined into a single composition comprising the two or more immunotherapeutic agents. For example, the present invention provides an immunotherapeutic composition comprising a PAP/GM-CSF fusion protein or conjugate, as described herein above, in combination with one or more additional immunotherapeutic such as, for example, a therapeutic antibody, an anti-cancer vaccine, and/or a cell-based therapeutic. Exemplary therapeutic antibodies include, but are not limited to, antibodies, such as monoclonal antibodies, that bind to Her-2/neu, CEA, CD20, CEA, VEGF, and/or other tumor-associated antigens.

An exemplary combined immunotherapeutic composition provided herein comprises a PAP/GM-CSF fusion protein in combination with an anti-VEGF (vascular endothelial growth factor) monoclonal antibody. For example, a suitable anti-VEGF antibody is the humanized murine monoclonal antibody Bevacizumab (Avastin™; Genentech, San Francisco, Calif.) that is known to be effective in inhibiting tumor angiogenesis.

Immunotherapeutic Methods

Within other embodiments, the present invention provides methods for inhibiting the growth of cancer cells that employ immunotherapeutic compositions as described herein above. These methods are based on the observation that cancer cells exhibiting a moderately to well-differentiated phenotype and corresponding growth characteristics are uniquely susceptible to immunotherapeutic treatment regimens. Thus, methods according to the present invention comprise the steps of: (a) determining in a cancer patient the grade of the cancer, (b) administering to the patient a therapeutically effective dose of an immunotherapeutic composition, and (c) monitoring the progression of the cancer.

By these methods, immunotherapeutic treatment regimens are employed in those instances in which the patient has a moderately to well-differentiated grade of cancer. A sample containing one or more cancer cells is isolated from the patient and the grade of those cancer cells is determined as described in detail above. Those patients having a moderately to well-differentiated grade of cancer are selected for treatment with an immunotherapeutic composition. A therapeutically effective dose of the immunotherapeutic composition is administered and the progression of the cancer is monitored to ascertain therapeutic efficacy. A reduction in tumor progression by 10%, 25%, or 50% indicates the effective treatment of the cancer by the methods of the present invention.

Alternative embodiments of the present invention provide immunotherapeutic treatment regimens comprising the administration of two or more immunotherapeutic agents. As exemplified herein, a suitable therapeutic treatment regimen comprises the step of administering a first immunotherapeutic agent comprising PAP/GM-CSF-pulsed dendritic cells (DC) and the step of administering a second immunotherapeutic agent comprising an anti-VEGF monoclonal antibody, such as bevacizumab. By these methods, the PAP/GM-CSF-pulsed DC may be administered to a patient simultaneously with administration of the anti-VEGF monoclonal antibody. Alternatively, the PAP/GM-CSF-pulsed DC may be administered independently from administration of the anti-VEGF monoclonal antibody. For example, within a specific embodiment of the present invention, PAP/GM-CSF-pulsed DC may be administered IV on weeks 0, 2, and 4 and bevacizumab may be administered IV on weeks 0, 2, 4 and every 2 weeks thereafter until an event such as toxicity, progressive disease, and/or development of metasteses.

Routes and frequency of administration of the immunotherapeutic compositions described herein, as well as dosage, will vary from individual to individual, and may be readily established using standard techniques. In general, the immunotherapeutic compositions may be administered by injection (e.g., intracutaneous, intramuscular, intravenous or subcutaneous), intranasally (e.g., by aspiration) or orally. Preferably, between 1 and 10 doses may be administered over a 52-week period. Alternate protocols may be appropriate for individual patients.

A suitable dose is an amount of a compound that, when administered as described above, is capable of promoting an anti-tumor immune response, and is at least 10-50% above the basal (i.e., untreated) level. Such response can be monitored by measuring the cytolytic effector cells capable of killing the patient's tumor cells ex vivo or anti-tumor antibodies in a patient. Such immunotherapeutic compositions should also be capable of causing an immune response that leads to an improved clinical outcome (e.g., more frequent remissions, complete or partial or longer disease-free survival) in treated patients as compared to untreated patients. In general, for immunotherapeutic compositions comprising one or more polypeptides, the amount of each polypeptide present in a dose ranges from about 25 mg to 5 mg per kg of host. Suitable dose sizes will vary with the size of the patient, but will typically range from about 0.1 mL to about 5 mL.

In general, an appropriate dosage and treatment regimen provides the immunotherapeutic composition in an amount sufficient to provide therapeutic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

Methods of Assessing Susceptibility of Cancer Cells to Immunotherapy

The present invention also provides methods for assessing the susceptibility of cancer cells to immunotherapeutic compositions. As discussed above, it was observed, as part of the present invention, that cancer cells exhibiting a moderately to well-differentiated phenotype are uniquely susceptible to immunotherapeutic treatment modalities.

Thus, the present invention provides methods of assessing in a cancer patient the susceptibility of the cancer to an immunotherapeutic composition comprising the steps of: (a) isolating from the patient a sample containing said cancer cell; and (b) determining the differentiation state of said cancer cell; wherein a moderate to well differentiated cancer grade indicates that the cancer cell is susceptible to treatment with an immunotherapeutic composition.

As discussed in detail herein above, differentiation state of a cancer cell may be defined by assessing its grade. Well-differentiated cancers are assigned a grade of 1, moderately differentiated cancers are assigned a grade of 2, and poorly differentiated cancers are assigned a grade of 3.

Exemplary cancers that are amenable to the methods of assessment according to the present invention include, for example, soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate. It will be appreciated, however, that the present methods of assessment are equally suited to any and all cancers for which there exist well established criteria for distinguishing well-differentiated and moderately differentiated cancer cells from poorly differentiated cancer cells.

Cancers identified as well-differentiated or moderately differentiated, as discussed above, are susceptible to treatment with any of the immunotherapeutic compositions disclosed herein or otherwise readily available in the art. In the specific case of prostate cancer, the present methods may be employed to identify patients having well-differentiated or moderately differentiated cells, as evidenced, for example, by a Gleason score of ≦7, and, consequently, who are susceptible to treatment with one of the immunotherapeutic compositions employing stimulated dendritic cells such as dendritic cells that are stimulated by a protein conjugate as disclosed herein. Within certain preferred embodiments, are provided methods of assessing the susceptibility to treatment of prostate cancer cells wherein the immunotherapeutic composition comprises a dendritic cell stimulated ex vivo with a PAP/GM-CSF fusion protein as disclosed herein above.

The following Example is offered by way of illustration and not by way of limitation.

### EXAMPLE 1

### Construction of PAP/GM-CSF Fusion Proteins

This example describes the construction of a cDNA encoding a PAP/GM-CSF fusion protein (PA2024) as previously disclosed in U.S. Pat. Nos. 5,976,546, 6,080,409, and 6,210,662.

Human PAP was cloned from the prostate carcinoma cell line LnCaP.FGC (American Type Culture Collection, Rockland Md.; “ATCC”). Synthetic oligonucleotide primers were custom synthesized according to standard methods by Keystone Labs (Menlo Park, Calif.). These primers were homologous to the 5′ end of the known PAP cDNA sequence presented herein as SEQ ID NO: 2. Hind III, Mun I or Xho I restriction sites were attached, according to the requirements of the particular expression vector used. On the 3′ end an oligonucleotide of was constructed that substituted a Bam HI restriction endonuclease site for stop codon of the PAP sequence and creating codons for glycine and serine. This Bam HI site was used to fuse the PAP cDNA (SEQ ID NO: 2) to the GM-CSF cDNA (SEQ ID NO: 4), which was cloned from peripheral blood mononuclear cells (PBMNC). At the GM-CSF 5′ end a Bam HI site was attached to an oligonucleotide homologous to the nucleotides that code for amino acids 18-23 in the GM-CSF sequence. The 3′ end of GM-CSF was generated with an oligonucleotide that ended after the in frame stop of GM-CSF and creates an Xba I cloning site.

Poly A+ RNA was isolated from cell line LnCaP.FGC and from PBMNC with the Micro Fast track kit (Invitrogen) according to the manual supplied by the manufacturer. The Poly A+ RNA was then reverse transcribed with the cDNA cycle kit (Invitrogen) according to procedures described in the accompanying manual. First strand cDNA was then subjected to 25 cycles of polymerase chain reaction (PCR) with the above described primers. The PCR products were cloned into the vectors pCR3 (Invitrogen) to create pCR3-PAP-GM, pCEP 4 (Invitrogen) to create pCEP4-PAP GM and into pBacPac 8 (Clontech) to create PAPHGM-BAC. The DNA sequences of the cloned constructs were confirmed using standard methods on a fluorescent sequencer Model ABI 373A (Applied Biosystems, Foster City, Calif.). The nucleotide sequence and the deduced amino acid sequences are presented as SEQ ID NO.: 6 and SEQ ID NO: 5, respectively.

pCR3 PAP-GM was electroporated into COS-7 cells (ATCC) for transient expression experiments. After it was confirmed that a protein of the predicted size, immunological identity and function could be expressed transiently in COS-7 and 293-EBNA (Invitrogen) cells stable transfectants were generated in the human embryonic kidney cell line 293-EBNA, using an episomal expression vector pCEP4 (Invitrogen, San Diego, Calif.). After electroporation and selection in hygromycin, recombinant clones were generated by plating the cells under limiting dilution conditions and screening for PAP bioactivity in the cellular tissue culture supernatants. The highest producing clones were adapted to protein-free media and grown in CellMax hollow fiber bioreactors (Gibco, Gaithersberg, Md.). Spent media from the cultures were collected, pooled and clarified by centrifugation. They were then passaged over an immunoaffinity column that was made by coupling the human PAP-specific monoclonal antibody ATCC HB8526 (ATCC) to a Sepharose resin. After washing, the bound material was eluted at low pH, neutralized and dialyzed against physiological buffer. The eluted fraction was analyzed by denaturing SDS-PAGE electrophoresis under reducing conditions. The resulting gel showed a single protein band at 75 kD which corresponds to the predicted size of fully glycosylated PAP/GM-CSF.

PAPHGM-BAC was also used to generate a recombinant Autographa californica nuclear polyhedrosis virus (AcNPV, baculovirus) by homologous recombination of PAPHGM-BAC with BacPAK6 viral DNA (Clontech, Palo Alto, Calif.). Reagents were used from the BacPAK baculovirus expression system (Clontech) and procedures were carried out essentially as described in the product manual. PAPHGM-BAC and BacPAK6 were cotransfected into SF21 cells (Clontech) by lipofection. The spent tissue culture supernatant was collected on day 5. It was titered onto fresh SF21 cells which were then grown in semisolid media for another 4 days. After the monolayers were stained with neutral red, viral plaques were identified and picked with a Pasteur pipet. Recombinant plaque-purified virus was eluted into fresh media and was then used to screen for production of PAP/GM-CSF in fresh SF21 cells. Positive plaques were identified and used to generate viral stocks and recombinant protein in subsequent rounds of infecting fresh SF21 cells. The media of production cultures were collected three days after infection. They were then processed as described for PAP/GM-CSF that was derived from 293-EBNA cells. Analysis of the immunoaffinity-purified protein revealed a single protein band at 64 kD after silver staining of an SDS-PAGE gel.

### EXAMPLE 2

### Bioactivity of PAP/GM-CSF Fusion Proteins

This Example describes the bioactivity of PAP/GM-CSF fusion proteins as previously disclosed in U.S. Pat. Nos. 5,976,546, 6,080,409, and 6,210,662.

PAP/GM-CSF fusion proteins from all expression systems described in Example 1 were analyzed for their ability to support the growth of GM-CSF dependenT-cell lines. They were also analyzed for enzymatic activity in acid phosphatase assays. Standard bioassays were used to determine the GM-CSF bioactivity.

GM-CSF Activity

The GM-CSF dependent human erythroleukemia cell line TF-1 (ATCC, Rockville, Md.) and the acute monocytic leukemia cell line AML-193 (ATCC) were used to analyze whether GM-CSF retains its bioactivity after fusion to PAP. The cell lines, which are routinely cultured in GM-CSF-containing media, were starved in regular media for 24 hours before the assay. They were plated at 1500 cells per well in triplicates in tissue culture medium. Test supernatants or recombinant GM-CSF as a positive control were added to the cells. Cells were cultured for 72 hours and were then pulsed for 4 hours with 1 microcurie tritiated thymidine per well to determine rate of DNA synthesis.

Acid Phosphatase Activity

The bioactivity of the second component of the fusion protein was determined in an enzymatic assay for acid phosphatase activity. Acid phosphatase was measured as the ability of the protein to hydrolyze para-nitrophenyl phosphate (PNPP) at acid pH. Briefly, the test liquid was diluted in 50 mM sodium citrate pH 4.8. pNPP was added to a final concentration of 2 mg/ml. After 30 minutes incubation at 37° C., an equal volume of 1 M NaOH was added to the reaction. Hydrolyzed pNPP under these conditions has a yellow color which can be quantified with a spectrophotometer at 405 nm.

### EXAMPLE 3

### Treatment of Hormone-Refractory Prostate Cancer Patients With an Immunotherapeutic Composition Comprising Antigen Presenting Cells Stimulated With a Prostatic Acid Phosphatase/GM-CSF Fusion Protein

This Example discloses the methodology employed in a randomized, placebo-controlled phase III clinical trial for the treatment of hormone refractory prostate cancer patients with an immunotherapeutic composition comprising APCs stimulated with a PAP/GM-CSF fusion protein (APC8015).

127 patients were selected for a phase III clinical trial based on the following criteria: (1) histologically confirmed adenocarcinoma of the prostate with evidence of disease progression despite androgen deprivation; (2) presence of Whitmore-Jewett stage D metastatic disease (Crawford et al., Urology 50(6):1027-1028 (1997)); (3) no cancer-related pain and no analgesics for pain; (4) tumor positive for PAP by immunohistology; (5) castration levels of testosterone<50 ng/dL; (6) PSA>5 ng/mL; (7) 6-months since conclusion of chemotherapeutic treatment regimen (or <3-months of CD4 count is >400); and (8) tumor progression after hormonal therapy. Tumor progression was assessed by radiographic progression either by CT or by at least two new hot-spots on bone scan and PSA progression was assessed by a level of at least 50% above level at time of castration therapy and stable or rising PSA on current therapy.

APC8015 was prepared fresh for each treatment course. Dendritic-cell precursors were harvested from the peripheral blood by a standard 1.5 to 2.0 blood volume mononuclear cell leukapheresis. Mobilization with a colony-stimulating factor was not required. The leukapheresis products were prepared at a local blood bank and transported to a Dendreon cell processing. Dendritic-cell precursors were collected by two sequential buoyant density centrifugation steps by a modification of the method of Hsu et al. Nat. Med. 2:52-58 (1996); Kundu et al., AIDS Res Hum. Retroviruses 14:51-560 (1998); and Peshwa et al., Prostate 36:129-138 (1998).

Briefly, the leukapheresis product was layered over a buoyant density solution (specific gravity=1.077 g/ml) and centrifuged at 1,000 g for 20 minutes to deplete erythrocytes and granulocytes. The interface cells were collected, washed, layered over a second buoyant density solution (specific gravity=1/065 g/ml), and centrifuged at 805 g for 30 minutes to deplete platelets and low-density monocytes and lymphocytes.

As described in detail above, PA2024, the target antigen for APC8015, is a fusion protein between huPAP and huGM-CSF. The fusion protein was cloned into a baculovirus system and expressed in Sf21 insecT-cells adapted to grow in serum-free media. PA2024 was purified by three sequential column chromatography steps to more than 95% homogeneity. Both protein components retained appropriate biologic activity, as demonstrated by enzymatic activity for PAP and growth promotion activity for GM-CSF. (See Example 2 for a description of PAP and GM-CSF activity assays).

The cell pellet, which contained dendritic-cell precursors, was washed and incubated in AIM media with 10 μg/ml of target antigen PA2024 (for APC8015 treated patients) or without target antigen PA2024 (for placebo treated patients). The culture medium did not contain serum or exogenous cytokines. After incubation for 40 hours at 37° C. in 5% CO₂ atmosphere, the cells were washed and formulated at the desired clinical dose in 250 ml of lactated Ringer's solution.

Criteria for releasing the immunotherapeutic APC8015 compositions for infusion included the following: (1) in-process sterility tests with no growth at 40 hours; (2) endotoxin less than 1.4 EU/ml; (3) CD54 expression greater than 3 SD above T=0 value; and (4) cell viability greater than 72%. In addition, phenotype was determined by flow cytometry (FACS) using monoclonal antibodies to CD4, CD8, CD54, CD56, CD66b, and CD86 (Becton Dickenson, San Jose Calif.; Coulter, Miami, Fla.). Additional tests, the results of which were available after infusion, were final product sterility and mycoplasma The final APC8015 or placebo products were transported back to the originating hospital at 4° C. and infused into the patients within 8 hours of formulation. APC8015 and placebo were infused separately, each over 30 minutes. Patients were not routinely premedicated before infusion. They were observed for 30 minutes after infusion and then discharged to home. Patients without prior orchiectomy continued on gonadal suppression with a lutenizing hormone-releasing hormone agonist.

For the 82 patients receiving APC8015 and the 45 patients treated with a placebo containing dendritic cells without prior stimulation with PA2024 PAP/GM-CSF fusion protein, leukapheresis was performed on days −2, 12, and 26 while respective infusions were performed on days 0, 14, and 28.

Patients were observed until objective disease progression or 1 year, whichever was first. Clinical endpoint was assessed by the following criteria: (1) disease progression by scans every 8 weeks and (2) onset of disease related pain. Serum PSA levels were measured every 4 weeks until disease progression. Time to progression was defined as the time from the day of registration until the day objective disease progression was documented. Patients who elected to come off study without objective disease progression (e.g., for increasing PSA) were considered to have disease progression at the time of study withdrawal. Primary endpoints included (1) objective disease progression as measured by bone scan, computerized tomography (CT) and magnetic resonance imaging (MRI) and (2) safety. Secondary endpoints included disease-related pain progression and response rates.

### EXAMPLE 4

### Therapeutic Efficacy of an Immunotherapeutic Composition Correlates With Prostate Cancer Differentiation State

This Example demonstrates that the therapeutic efficacy of an immunotherapeutic composition comprising APCs stimulated with a PAP/GM-CSF fusion protein (i.e. APC8015) correlates with the differentiation state of the prostate cancer cells.

Prior to initiating an immunotherapeutic treatment regimen with APC8015 or placebo, as described above, patients were assessed for baseline disease characteristics. To determine the differentiation state of prostate cancer cells, prostate tissue samples were isolated from each patient and subjected to analysis by the Gleason scoring methodology as described in Gleason, Urologic Pathology: The Prostate, pp. 171-197 (Tappenhaum, ed., Lee & Fehiger, Philadelphia, Pa., 1977). The results of this assessment are presented in Table 1.

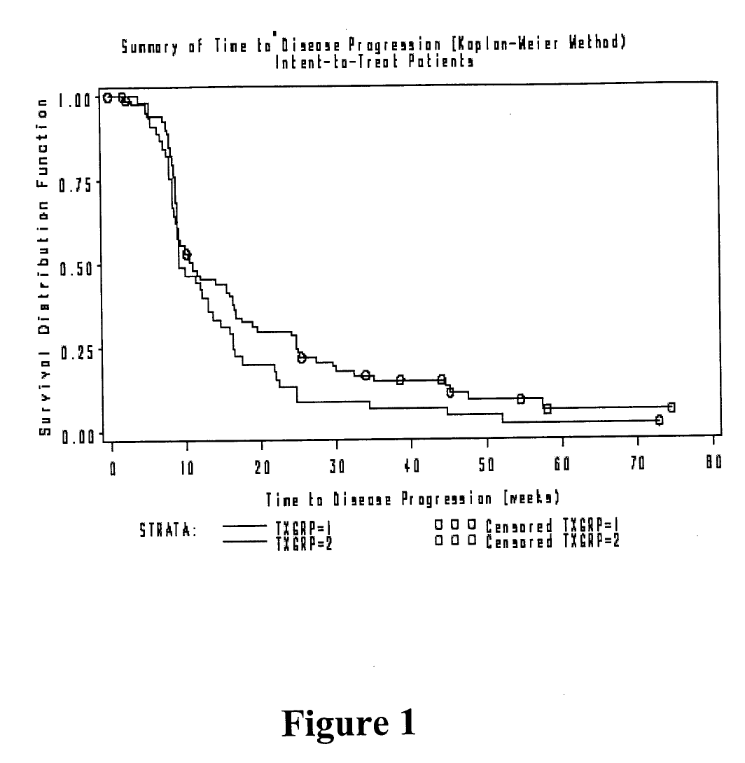

Time to objective disease progression was defined as progression on bone scan or x-ray or clinical deterioration and the data were subjected to statistical analysis by the Kaplan-Meier methodology. PSA was not used to determine disease progression. As shown in Table 2, the median time to disease progression for the patient population treated with APC8015 was 11.0 weeks whereas the median time to disease progression for the patient population treated with placebo was 9.1 weeks. Table 3 and FIG. 1 show the percentage of progression free survival as a function of time following administration of APC8015 or placebo. The p-value derived by comparison of the time to disease progression curves for the two populations was 0.085.

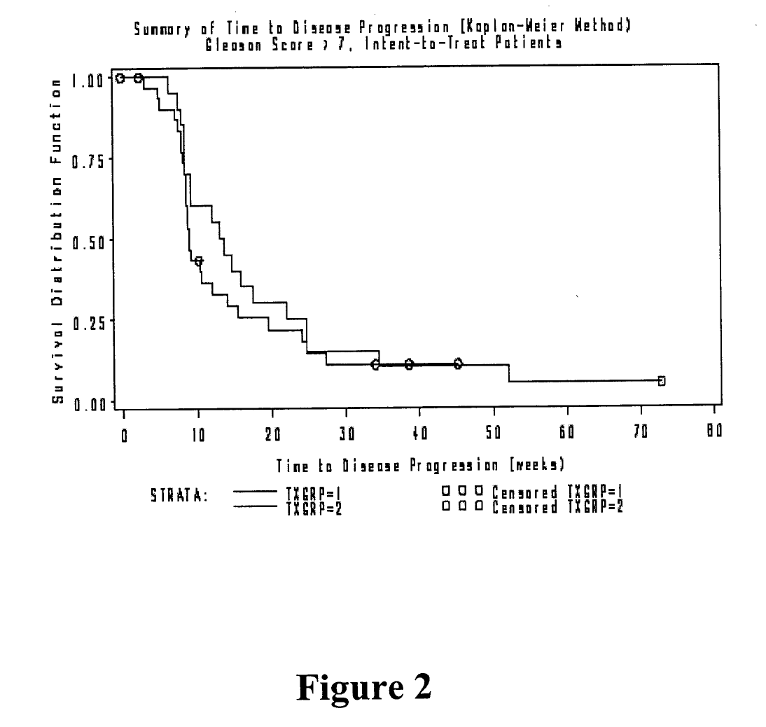

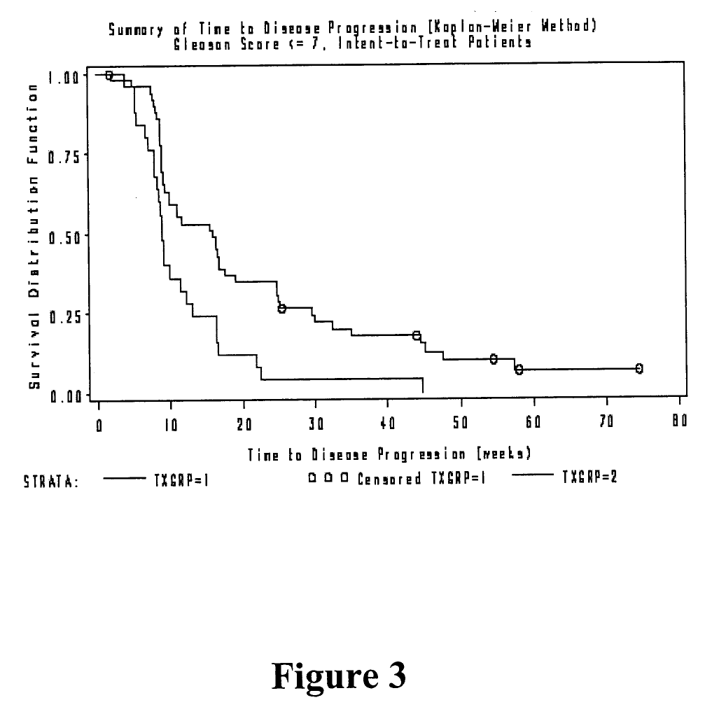

Tables 4 and 6 disclose, respectively, the time to objective disease progression for patients exhibiting poorly differentiated prostate cancer cells (Gleason score≧8) and for patients exhibiting moderately to well-differentiated prostate cancer cells (Gleason score≦7). Table 5 and FIG. 2 and Table 7 and FIG. 3 show the percentage of progression free survival as a function of time following administration of APC8015 or placebo to patient populations exhibiting poorly differentiated and moderately to well-differentiated cancer cells, respectively.

These data demonstrated that patients having poorly differentiated prostate cancer cells were refractory to treatment with APC8015 as evidenced by the absence of a statistically significant difference (p-value=0.431) in time to objective disease progression for the patient population treated with APC8015 as compared to the patient population treated with the placebo. In contrast, the results obtained for patients exhibiting moderately to well-differentiated prostate cancer cells (having a Gleason score of ≦7) show that such patients were susceptible to treatment with an immunotherapeutic composition as evidenced by the high degree of statistical significance (p-value=0.002) in time to objective disease progression for the patient population treated with APC8015 as compared to the patient population treated with the placebo.

TABLE 1
Baseline Disease Characteristics

| Gleason Score | APC8015 | Placebo |
|---|---|---|
| (range = 2-10) | (N = 82) | (N = 45) |
| Median | 7.0 | 7.0 |
| Score, n (%) | | |
| ≦6 | 22 (26.8) | 7 (15.6) |
| 7 | 28 (34.1) | 18 (40.0) |
| ≧8 | 32 (39.0) | 20 (44.4) |

TABLE 2
Time to Objective Disease Progression
(Kaplan-Meier Method)
Primary Efficacy Analysis
Intent-to-Treat Patients

| Quartile Estimates | APC8015 | | Placebo | |
|---|---|---|---|---|
| (95% Confidence Interval) | (N = 82) | | (N = 45) | |
| 25% | 8.7 | weeks | 8.3 | weeks |
| | (8.1, 8.9) | | (7.1, 8.9) | |
| 50% | 11.0 | weeks | 9.1 | weeks |
| (Median) | (9.1, 16.3) | | (8.7, 13.1) | |
| 75% | 24.7 | weeks | 16.3 | weeks |
| | (16.7, 32.4) | | (13.1, 22.4) | |
| Range | 2.1-57.4 | weeks | 3.9-52.1 | weeks |
| (Excluding Censored Values) | | | | |
| Range | 0*-74.6 | weeks | 3.9-72.9 | weeks |
| (Including Censored Values) | | | | |

p-value^{a} = 0.085

^{a}p-value compares the time to disease progression curves of the treatment groups using the log-rank test
*Censored observation

TABLE 3
Progression Free Survival Rate Estimate
(95% Confidence Interval)

| | APC8015 | Placebo | Difference |
|---|---|---|---|
| | (N = 82) | (N = 45) | (APC8015 − Placebo) |
| 12 Weeks | 45.4 | 44.4 | 0.9 |
| | (34.4, 56.4) | (29.9, 59.0) | (−17.1, 19.0) |
| 24 Weeks | 29.8 | 13.3 | 16.5 |
| | (19.7, 40.0) | (3.4, 23.3) | (2.5, 30.5) |
| 36 Weeks | 15.0 | 6.7 | 8.4 |
| | (6.9, 23.1) | (0.0, 14.0) | (−2.3, 19.0) |
| 48 Weeks | 9.0 | 4.4 | 4.6 |
| | (1.8, 16.2) | (0.0, 10.5) | (−4.1, 13.2) |
| 72 Weeks | 6.0 | 2.2 | 3.8 |
| | (0.0, 12.8) | (0.0, 6.5) | (−2.9, 10.5) |

TABLE 4
Time to Objective Disease Progression
(Kaplan-Meier Method)
Gleason Score > 7
Intent-to-Treat Patients

| Quartile Estimates | APC8015 | | Placebo | |
|---|---|---|---|---|
| (95% Confidence Interval) | (N = 82) | | (N = 45) | |
| 25% | 8.1 | weeks | 8.4 | weeks |
| | (7.1, 8.7) | | (8.0, 13.1) | |
| 50% | 9.0 | weeks | 13.4 | weeks |
| (Median) | (8.6, 12.0) | | (8.4, 22.1) | |
| 75% | 19.6 | weeks | 23.4 | weeks |
| | (10.4, 27.4) | | (13.6, 34.4) | |
| Range | 3-27.4 | weeks | 6.4-52.1 | weeks |
| (Excluding Censored Values) | | | | |
| Range | 0*-45.3* | weeks | 6.4-72.9* | weeks |
| (Including Censored Values) | | | | |

p-value^{a} = 0.431

^{a}p-value compares the time to disease progression curves of the treatment groups using the log-rank test
*Censored observation

TABLE 5
Progression Free Survival Rate Estimate
(95% Confidence Interval)
Gleason Score > 7

| | APC8015 | Placebo | Difference |
|---|---|---|---|
| | (N = 82) | (N = 45) | (APC8015 − Placebo) |
| 12 Weeks | 32.5 | 60.0 | −27.5 |
| | (15.5, 49.5) | (38.5, 81.5) | (−45.0, −10.0) |
| 24 Weeks | 21.7 | 25.0 | −3.3 |
| | (6.5, 36.8) | (6.0, 44.0) | (−18.8, 12.1) |
| 36 Weeks | 10.8 | 10.0 | 0.8 |
| | (0.0, 22.3) | (0.0, 23.1) | (−10.2, 11.9) |
| 48 Weeks | 10.8 | 10.0 | 0.8 |
| | (0.0, 22.3) | (0.0, 23.1) | (−10.2, 11.9) |
| 72 Weeks | 10.8 | 5.0 | 5.8 |
| | (0.0, 22.3) | (0.0, 14.6) | (−3.4, 15.1) |

TABLE 6
Time to Objective Disease Progression
(Kaplan-Meier Method)
Gleason Score ≦ 7
Intent-to-Treat Patients

| | | |
|---|---|---|
| Quartile Estimates | APC8015 | Placebo |
| (95% Confidence Interval) | (N = 82) | (N = 45) |

| 25% | 8.9 | weeks | 8.0 | weeks |
|---|---|---|---|---|
| | (8.7, 10.0) | | (5.6, 8.9) | |
| 50% | 16.0 | weeks | 9.0 | weeks |
| (Median) | (9.3, 19.0) | | (8.0, 12.3) | |
| 75% | 29.6 | weeks | 13.1 | weeks |
| | (17.6, 45.3) | | (9.1, 16.6) | |
| Range | 2.1-57.4 | weeks | 3.9-44.9 | weeks |
| (Excluding Censored | | | | |
| Values) | | | | |
| Range | 1.9*-74.6* | weeks | 3.9-44.9 | weeks |
| (Including Censored | | | | |
| Values) | | | | |

p-value^{a} = 0.002

^{a}p-value compares the time to disease progression curves of the treatment groups using the log-rank test
*Censored observation

TABLE 7
Progression Free Survival Rate Estimate
(95% Confidence Interval)
Gleason Score ≦ 7

| | APC8015 | Placebo | Difference |
|---|---|---|---|
| | (N = 82) | (N = 45) | (APC8015 − Placebo) |
| 12 Weeks | 53.1 | 32.0 | 21.1 |
| | (39.1, 67.0) | (13.7, 50.3) | (3.7, 38.5) |
| 24 Weeks | 34.7 | 4.0 | 30.7 |
| | (21.4, 48.0) | (0.0, 11.7) | (18.9, 42.5) |
| 36 Weeks | 17.7 | 4.0 | 13.7 |
| | (6.8, 28.5) | (0.0, 11.7) | (3.6, 23.7) |
| 48 Weeks | 10.1 | 0.0 | 10.1 |
| | (1.1, 19.1) | (0.0, 0.0) | (3.6, 16.6) |
| 72 Weeks | 6.7 | 0.0 | 6.7 |
| | (0.0, 14.8) | (0.0, 0.0) | (1.3, 12.2) |

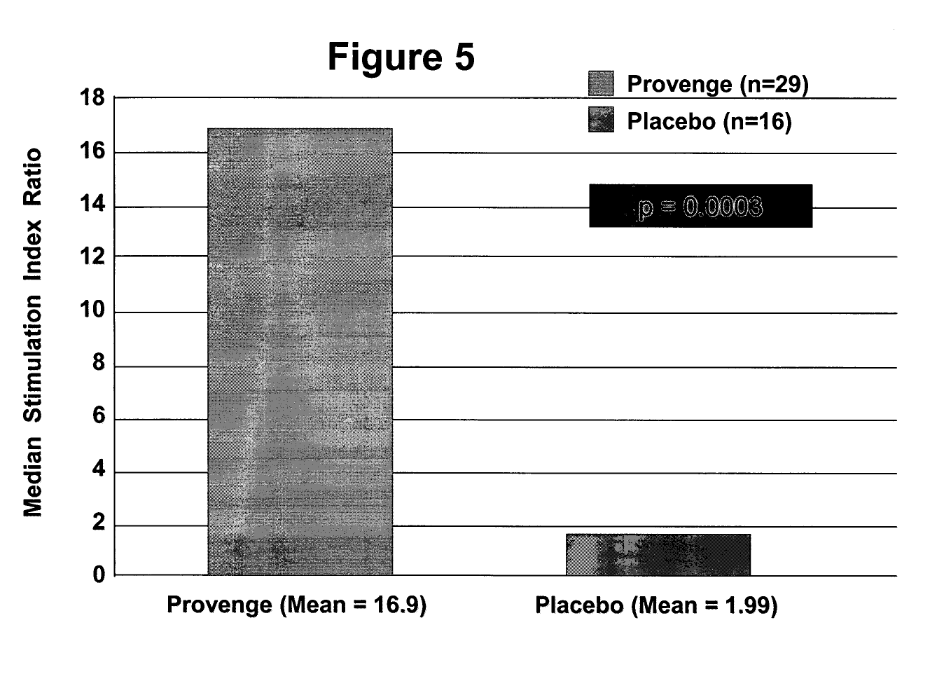

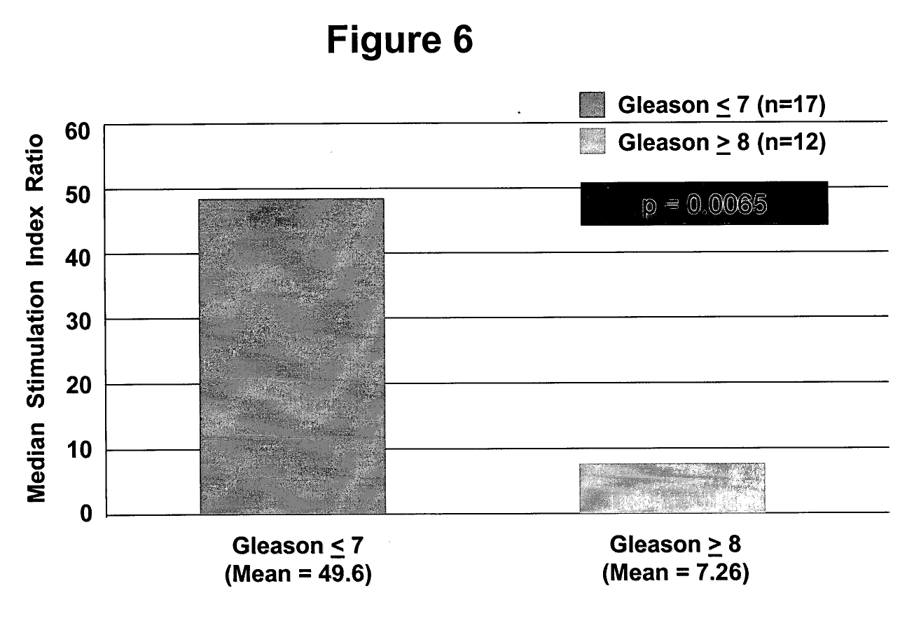

FIG. 5 presents data demonstrating that patients receiving APC8015 exhibit a statistically significant enhancement in median T-cell mediated immune response as compared to patients receiving placebo (p-value=0.0003). FIG. 6 presents data demonstrating that the patient population having a Gleason score of ≦7 and receiving APC8015 exhibit a statistically significant enhancement in median T-cell mediated immune response as compared to a patient population having a Gleason score of ≧8 and receiving APC8015 (p-value=0.0065).

### EXAMPLE 5

### Time to Onset of Disease-Related Pain

This Example demonstrates that the time to onset of disease-related pain in patients receiving an immunotherapeutic composition comprising APCs stimulated with a PAP/GM-CSF fusion protein (i.e. APC8015) vs. placebo is prolonged in prostate cancer patients having a Gleason score of ≦7 while the time to onset of disease-related pain is virtually unaffected in prostate cancer patients having a Gleason score of ≧8.

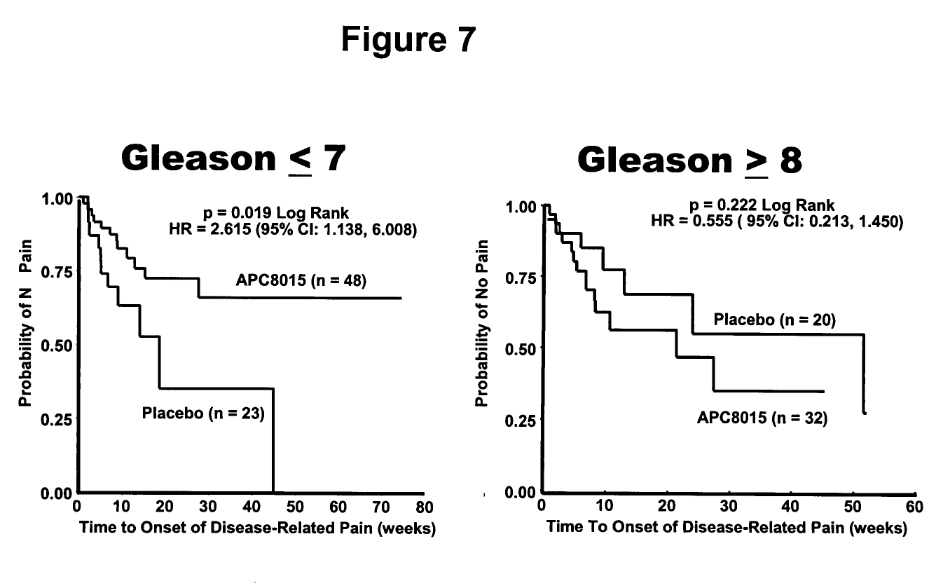

Disease-related pain was defined as pain that has a quality and consistency of cancer-related pain, occurred since enrolling in the study, and the location of the pain correlated with a site of disease that was objectively confirmed by radiographic means. Time to onset of disease related pain is the time from patient randomization to the onset of pain. Pain was measured in 2 ways: patient completed weekly pain logs based on the Wisconsin Brief Inventory, a well validated pain assessment tool, and physician assessment during clinic visits. Blinded external reviewers (i.e. not the physician who saw the patient) reviewed the evidence. Pain was only deemed cancer related when the location correlated with an imaged site of disease. The pain-data were analyzed in a time-to-event analysis using statistical analysis by the Kaplan-Meier method. As shown in FIG. 7, the median time to onset of disease-related pain for a patient population having a Gleason score of ≦7 (moderately to well-differentiated prostate cancer cells) treated with placebo was 18.7 weeks. The median time to onset of pain had not yet been reached at the end of the study in the APC8015 group. The difference between APC8015 and placebo was statistically significant (log rank p=0.016). In contrast, the difference between APC8015 and placebo in median time to onset of disease-related pain for a patient population having a Gleason score of ≧8 (poorly differentiated prostate cancer cells) was not significant (p=0.304).

These data demonstrate that patients having poorly differentiated prostate cancer cells exhibited an onset of disease-related pain that is virtually unaffected by treatment with APC8015 as evidenced by the absence of a statistically significant difference (log rank p=0.304) in time to onset of disease-related pain for the patient population treated with APC8015 as compared to the patient population treated with the placebo. In contrast, the results obtained for patients exhibiting moderately to well-differentiated prostate cancer cells (having a Gleason score of ≦7) show that such patients benefited from treatment with APC8015 as evidenced by the high degree of statistical significance (log rank p=0.016) in time to onset of disease-related pain for the patient population treated with APC8015 as compared to the patient population treated with the placebo.

Furthermore, data presented in Table 8 demonstrate that APC8015 was favorably tolerated by prostate cancer patients as compared to an equivalent patient population receiving placebo (occurring in ≧10% of patients; p-value=≦0.05).

TABLE 8
APC8015 is Well-tolerated by Prostate Cancer Patients

| | APC8015 | Placebo | Median Duration |
|---|---|---|---|
| Event [n(%)] | (n = 82) | (n = 45) | (days) |
| Rigor | 49 (59.8) | 3 (6.7) | 1.0 |
| Pyrexia | 24 (29.3) | 1 (2.2) | 2.0 |
| Headache NOS | 11 (13.4) | 1 (2.2) | 1.0 |
| Paresthesia | 11 (13.4) | 1 (2.2) | 2.0 |
| Dyspnea | 9 (11.0) | 1 (2.2) | 1.0 |

Number (%) of patients with adverse effects

### EXAMPLE 6

### PAP-GMCSF-Pulsed Dendritic Cells in Combination With Bevacizumab in Patients With Serologic Progression of Prostate Cancer After Local Therapy

This Example discloses the efficacy of a combined immunotherapeutic treatment regimen that includes the administration of PAP/GM-CSF-pulsed dendritic cells in conjunction with the administration of a humanized anti-VEGF monoclonal antibody Bevacizumab in patients having a serological progression of prostate cancer.

Briefly, patients with androgen-dependent (hormone-sensitive) prostate cancer and prior definitive surgical or radiation therapy with non-metastatic, recurrent disease as manifested by a rising PSA between 0.4 ng/ml and 6.0 ng/ml were enrolled in a phase II clinical trial. PAP/GM-CSF-pulsed patient DCs were given intravenously (IV) on weeks 0, 2 and 4. Bevacizumab (10 mg/kg) was given IV on weeks 0, 2, 4 and every 2 weeks thereafter until toxicity or progressive disease, defined as a doubling of the baseline PSA value (to at least >4 ng/ml) or development of metastases. T-cell proliferation, cytokine production in response to PAP and DC costimulatory/activation marker expression was assayed. Table 9 shows the inclusion and exclusion criteria for patient eligibility into the phase II clinical trial.

TABLE 9

| | Major Eligibility Criteria |
|---|---|
| Inclusion | Histologic diagnosis of adenocarcinoma of the |
| | prostate |
| | Prior definitive therapy for primary prostate cancer |
| | consisting of: |
| | External beam radiotherapy; |
| | Brachytherapy ± pelvic XRT; or |
| | Radical prostatectomy (RP) ± adjuvant |
| | or salvage XRT |
| | Therapeutic PSA response to primary therapy <1.0 ng/ml |
| | post XRT or <0.4 ng/ml post RP |
| | Elevated PSA (between 0.4 ng/ml and 6.0 ng/ml) |
| | which has risen serially from baseline on two |
| | determinations at least one week apart |
| | No clinical evidence of local recurrence or metastases |
| | Estimated life expectancy of at least 12 months and |
| | ECOG performance status of 0 or 1 |
| Exclusion | Cryosurgery as the only definitive therapy |
| | Current systemic steroid therapy |
| | Prior hormonal therapy for treatment of progressive |
| | disease. (Prior hormonal therapy used as |
| | adjuvant/neoadjuvant tx. was permitted, but the last |
| | day of effective androgen deprivation must have been |
| | at least 3 months prior to study entry |
| | Prior chemotherapy, immunotherapy, or other |
| | experimental agents for prostate |
| | History of deep vein thrombosis, bleeding disorder or |
| | current/recent use of oral or parenteral anticoagulants |
| | or aspirin |

Patient Disposition

14 patients enrolled for this phase II clinical trial. One patient withdrew consent, six patients were removed from the study with PSA failure after a median of 28 weeks of treatment (range 12-48 weeks), and one patient was removed from the study at week 60 with a stable PSA secondary to grade 3 CHF, possibly related to protocol treatment. Six patients remained on the treatment with a stable PSA at a median of 28 weeks (range 2 to 60 weeks). Of the patients have been treated, the median age was 61 (range 60-76). Gleason scores for 11 of the patients were 5 (2 patients), 6 (5 patients), 7 (2 patients), and 8 (2 patients).

Administration of PAP/GM-CSF-pulsed DC and Bevacizumab

Dendritic cells (DC) precursors were harvested from the peripheral blood on day 1 or weeks 0, 2 and 4 by four-hour leukapheresis. DC precursors were isolated from the leukapheresis product by buoyant density centrifugation. Precursor cells were cultured with PAP/GM-CSF for 40 hours. Patients received the maximal manufacturable dose of PAP/GM-CSF-pusled DC, ˜1.2×10⁹ nucleated cells/m², by IV infusion over 30 minutes on day 3 or weeks 0, 2, and 4. Thirty minutes prior to infusion, patients were premedicated with oral acetaminophen 650 mg and diphenhydramine 50 mg. Bevacizumab (10 mg/kg IV over 90 minutes) was administered on day 3 of weeks 0, 2 and 4 (following PAP/GM-CSF-pulsed DC infusion) and every 2 weeks thereafter.

PSA Modulation

PSA changes were monitored for each of the patients. A decrease in PSA levels was detected in three patients (12% decrease, 33% decrease, 64% decrease). Median baseline PSA was 1.88 ng/ml (range 0.5-5.08 ng/ml). Of the nine patients evaluable for response, three had a decrease in the PSA doubling time (PSADT; estimated using the slope of the PSA versus time) including one patient who demonstrated a PSA decline from 2.74 ng/ml at study entry to 1.43 ng/ml (after having reached a PSA value of 5.6 ng/ml at week 12).

Table 10 summarizes the pre-treatment and on-treatment PSA kinetics. The PSA doubling time (PSADT) both pre and on-treatment was estimated using the relationship of Ln 2 divided by the slope of the PSA versus time curve. PSADT was calculated for patients with a positive slope of the on-treatment PSA versus time curve. Three patients could not have on-treatment PSADT calculated because of declining PSA values. Three patients had an increase in PSADT and three patients had no change in PSADT. No patient had objective disease progression.

TABLE 10

| Characteristic | Pre-treatment | On-treatment |
|---|---|---|
| Maximum PSA (n = 12) | | |
| Median | 2.56 ng/ml | |
| Range | (0.7, 5.08) | |
| PSA Nadir (n = 12) | | |
| Median | | 2.31 ng/ml |
| Range | | (0.47, 5.08) |
| PSA Doubling Time (n = 9) | | |
| Median | 8.2 months | 21.4 months |
| Mean | 9.4 months | 22.5⁺ months |
| Range | (1.0-32.6) | (2.0-76+) |

T-cell Proliferation

Peripheral blood lymphocytes were isolated from each patient at baseline, week 8 and week 12. In a 96 well U bottom plate, 1×10⁵ PBMCs were added to PAP/GM-CSF titrated in RPMI-10% human-sera media. Pokeweed mitogen was used as a positive control. The assay was incubated in a 37° C. water-jacket incubator at 5% CO₂ for 6 days. The assay was pulsed with ³H-thymidine (Amersham, Piscataway, N.J.) for the last 18 hours of incubation then harvested to filter mats using a Tomtec plate harvester. After the addition of scintillation cocktail (Perkin Elmer/Wallac), the assay was counted using a Wallac beta scintillation counter. Read-out was reported in counts per minute (CPM).

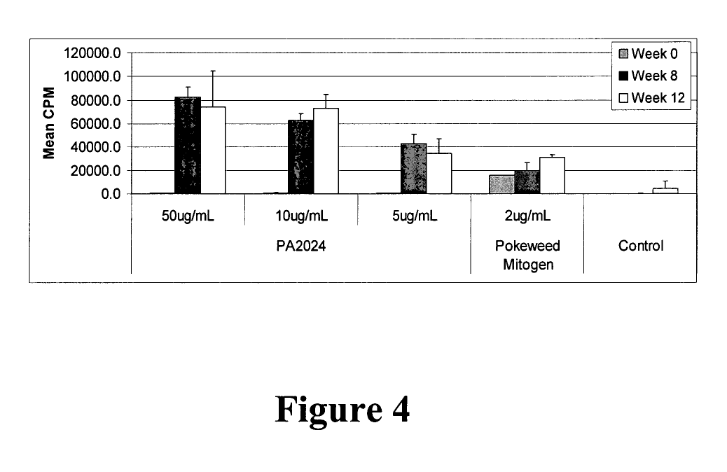

The results of these experiments are shown in FIG. 4 that shows T-cell proliferation of a representative patient in response to varying concentrations of PAP/GM-CSF (from 2 μg/ml to 50 μg/ml). This patient has an ongoing PSA decline of >50%. Two of four patients tested had demonstrable increased T-cell proliferation in response to PAP/GM-CSF at week 8 and 12.

IFN-γ ELISPOT

Wells of multiscreen-HA plates were coated overnight at 4° C. with 100 μl of anti-human IFNγ antibody at 15 μg/ml in D-PBS. Plates were then washed with PBST and blocked with 200 μl D-PBS+10% HS for 2 hours at 37° C. 3×10⁵ PBMCs were added with PAP/GM-CSF titrated in RPMI−10% HS. The assay was incubated at 37° C. for 40-48 hours. After 2 days, cells and antigen were washed from the plate using PBST. 100 μl of the detection antibody, biotinylated anti-human IFNγ was added to wells at 1 μg/ml in PBST. Plates were washed 6-times with PBST and 100 μl of StreptAvidin Alkaline Phosphatase (MabTech, Mariemont, Ohio) diluted 1:1000 in PBST was added to assay wells. The assay was incubated for 1.5 hours and then washed 6-times with PBST. 1-step BCIP/NBT solution was added to wells at 100 μl per well and incubated for 12 minutes to develop spots. Plates were scanned and spots counted using an ImmunoSpot Analyzer and software.

PAP/GM-CSF specific IFN-γ production was measured from patients using ELISPOT analysis at different PAP/GM-CSF concentrations. Three of three patients tested had demonstrable increased IFN-γ-producing T-cells at week 8 compared to baseline. One patient had insufficienT-cells for analysis.

These data demonstrate that the combination of PAP/GM-CSF and bevacizumab is effective, i.e. has PSA modulating activity, in the treatment of serologically-progressed prostate cancer. Immunologic analyses demonstrated a PAP/GM-CSF-specific immune response generated from the therapeutic treatment regimen and that the combination immunotherapy with PAP/GM-CSF and anti-VEGF antibody is safe and well-tolerated.

Although the present invention has been described in some detail by way of illustration and example for purposes of clarity of understanding, changes and modifications can be carried out without departing from the scope of the invention, which is intended to be limited only by the scope of the appended claims.

What is claimed is:

1. An immunotherapeutic composition, comprising activated, isolated antigen presenting cells (APCs), wherein said APCs are obtained from a patient diagnosed with a cancer having a moderate to well differentiated cancer grade and wherein said APCs are stimulated by exposure ex vivo to a tumor-associated antigen (TAA).

2. The immunotherapeutic composition of claim 1 wherein said tumor-associated antigen (TAA) is a tumor-specific antigen.

3. The immunotherapeutic composition of claim 1 wherein said tumor-associated antigen (TAA) is a component of a protein conjugate comprising an N-terminal moiety and a C-terminal moiety.

4. The immunotherapeutic composition of claim 1 wherein said APCs are dendritic cells (DCs).

5. The immunotherapeutic composition of claim 1 wherein said cancer is selected from the group consisting of soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate.

6. The immunotherapeutic composition of claim 1 wherein said cancer is prostate cancer.

7. The immunotherapeutic composition of claim 6 wherein said cancer grade corresponds to a Gleason score of ≦7.

8. The immunotherapeutic composition of claim 7 wherein said patient is not refractory to hormone ablation therapy.

9. The immunotherapeutic composition of claim 3 wherein said N-terminal moiety is an APC binding protein and said C-terminal moiety is a tumor-associated antigen (TAA).

10. The immunotherapeutic composition of claim 3 wherein said C-terminal moiety is an APC binding protein and said N-terminal moiety is a tumor-associated antigen (TAA).

11. The immunotherapeutic composition of claim 3 wherein said protein conjugate is a fusion protein.

12. The immunotherapeutic composition of claim 11 wherein said fusion protein further comprises, between said N-terminal moiety and said C-terminal moiety, a linker peptide.

13. The immunotherapeutic composition of claim 6 wherein said N-terminal moiety or said C-terminal moiety comprises a sequence having at least 70% sequence identity with the sequence depicted in SEQ ID NO: 1 (huPAP).

14. The immunotherapeutic composition of claim 6 wherein said N-terminal moiety or said C-terminal moiety comprises a sequence having at least 80% sequence identity with the sequence depicted in SEQ ID NO: 1 (huPAP).

15. The immunotherapeutic composition of claim 6 wherein said N-terminal moiety or said C-terminal moiety comprises a sequence having at least 90% sequence identity with the sequence depicted in SEQ ID NO: 1 (huPAP).

16. The immunotherapeutic composition of claim 6 wherein said N-terminal moiety or said C-terminal moiety comprises the sequence depicted in SEQ ID NO: 1 (huPAP).

17. The immunotherapeutic composition of any one of claims 3 through 16 wherein said C-terminal moiety or said N-terminal moiety is at least 70% identical to the sequence depicted in SEQ ID NO: 3 (huGM-CSF).

18. The immunotherapeutic composition of any one of claims 3 through 16 wherein said C-terminal moiety or said N-terminal moiety is at least 80% identical to the sequence depicted in SEQ ID NO: 3 (huGM-CSF).

19. The immunotherapeutic composition of any one of claims 3 through 16 wherein said C-terminal moiety or said N-terminal moiety is at least 90% identical to the sequence depicted in SEQ ID NO: 3 (huGM-CSF).

20. The immunotherapeutic composition of any one of claims 3 through 16 wherein said C-terminal moiety or said N-terminal moiety is the sequence depicted in SEQ ID NO: 3 (huGM-CSF).

21. A method of treating a cancer patient with an immunotherapeutic composition said patient having a cancer with moderately to well-differentiated cancer cells, said method comprising the steps of:
(a) determining in said patient the differentiation state of said cancer cells wherein the presence of moderately to well-differentiated cancer cells indicates a patient susceptible to treatment with an immunotherapeutic composition; and
(b) administering to said patient a therapeutically effective dose of an immunogenic composition, wherein a reduction of 10% indicates an effective treatment of said cancer.

22. The method of claim 21 wherein said immunotherapeutic composition is the immunotherapeutic composition of any one of claims 1-18.

23. A method of inhibiting growth of a cancer cell in a patient having a moderate to well differentiated cancer grade, said method comprising the steps of:
(a) determining in said patient the grade of said cancer cell wherein a moderate to well differentiated cancer grade indicates a patient susceptible to treatment;
(b) isolating antigen presenting cells (APCs) from a patient having a moderate to well differentiated cancer grade;
(c) stimulating said APCs by exposure ex vivo to an immunotherapeutic composition comprising a protein conjugate comprising an N-terminal moiety and a C-terminal moiety, wherein said APCs are effective to activate T-cells to produce a cytotoxic cellular response against either said N-terminal moiety or said C-terminal moiety and wherein the level of said T-cell activation is higher than that produced by said APCs when exposed exclusively to said N-terminal moiety or to said C-terminal moiety; and
(d) administering to said patient a therapeutically effective dose of said stimulated APCs, wherein a reduction of 10% indicates an effective treatment of said cancer.

24. The method of claim 23 wherein said cancer is selected from the group consisting of soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate.

25. The method of claim 24 wherein said cancer is prostate cancer.

26. The method of claim 25 wherein said cancer grade is determined by Gleason score and wherein said Gleason score is ≦7.

27. The method of claim 23 wherein said immunotherapeutic composition is the immunotherapeutic composition of any one of claims 3-18.

28. A method of assessing in a cancer patient the susceptibility of the cancer to an immunotherapeutic composition, said method comprising the steps of:
(a) isolating from said patient a sample containing said cancer cell; and
(b) determining the differentiation state of said cancer cell;
wherein a moderate to well differentiated cancer grade indicates that said cancer cell is susceptible to treatment with an immunotherapeutic composition.

29. The method of claim 28 wherein said cancer is selected from the group consisting of soft tissue sarcomas, lymphomas, and cancers of the brain, esophagus, uterine cervix, bone, lung, endometrium, bladder, breast, larynx, colon/rectum, stomach, ovary, pancreas, adrenal gland and prostate.

30. The method of claim 28 wherein said immunotherapeutic composition is an immunotherapeutic composition of any one of claims 1-20.
